(19) United States
(12) Patent Application Publication
(10) Pub. No.: US 2019/0211330 A1
(43) Pub. Date: Jul. 11, 2019

(54) COMPOSITIONS AND METHODS FOR TREATMENT OF SPINAL MUSCULAR ATROPHY

(71) Applicants: Ionis Pharmaceuticals, Inc., Carlsbad, CA (US); Cold Spring Harbor Laboratory, Cold Spring Harbor, NY (US)

(72) Inventors: Yimin Hua, Miami, FL (US); Adrian R. Krainer, East Northport, NY (US); Frank Rigo, Carlsbad, CA (US); C. Frank Bennett, Carlsbad, CA (US)

(73) Assignees: Ionis Pharmaceuticals, Inc., Carlsbad, CA (US); Cold Spring Harbor Laboratory, Cold Spring Harbor, NY (US)

(21) Appl. No.: 16/178,129

(22) Filed: Nov. 1, 2018

Related U.S. Application Data
(63) Continuation of application No. 14/129,029, filed on Jul. 29, 2014, now abandoned.
(60) Provisional application No. 61/501,135, filed on Jun. 24, 2011.

(51) Int. Cl. C12N 15/113; A61K 38/27; A61B 17/00; A61K 38/30

(52) U.S. Cl. CPC C12N 15/113; A61K 38/27; A61B 2017/22072; A61K 38/30; C12N 2310/11; A61B 17/0057

(57) ABSTRACT

Disclosed herein are compounds, compositions and methods for treatment of diseases and disorders, including spinal muscular atrophy.

The present application is being filed along with a Sequence Listing in electronic format. The Sequence Listing is provided as a file entitled CORE0096USC1SEQ_ST25.txt, created Nov. 1, 2018, which is 8 Kb in size. The information in the electronic format of the sequence listing is incorporated herein by reference in its entirety.

### BACKGROUND OF THE INVENTION

Proximal spinal muscular atrophy (SMA) is a genetic, neurodegenerative disorder characterized by the loss of spinal motor neurons. SMA is an autosomal recessive disease of early onset and is currently the leading cause of death among infants. The severity of SMA varies among patients and has thus been classified into three types. Type I SMA is the most severe form with onset at birth or within 6 months and typically results in death within 2 years. Children with type I SMA are unable to sit or walk. Type II SMA is the intermediate form and patients are able to sit, but cannot stand or walk. Patients with type III SMA, a chronic form of the disease, typically develop SMA after 18 months of age (Lefebvre et al., Hum. Mol. Genet., 1998, 7, 1531-1536).

The molecular basis of SMA is caused by the loss of both copies of survival motor neuron gene 1 (SMN1), which may also be known as SMN Telomeric, a protein that is part of a multi-protein complex thought to be involved in snRNP biogenesis and recycling. A nearly identical gene, SMN2, which may also be known as SMN Centromeric, exists in a duplicated region on chromosome 5q13 and modulates disease severity. Expression of the normal SMN1 gene results solely in expression of survival motor neuron (SMN) protein. Although SMN1 and SMN2 have the potential to code for the same protein, SMN2 contains a translationally silent mutation at position +6 of exon 7, which results in inefficient inclusion of exon 7 in SMN2 transcripts. Thus, the predominant form of SMN2 is a truncated version, lacking exon 7, which is unstable and inactive (Cartegni and Krainer, Nat. Genet., 2002, 30, 377-384). Expression of the SMN2 gene results in approximately 10-20% of the SMN protein and 80-90% of the unstable/non-functional SMNdelta7 protein. SMN protein plays a well-established role in assembly of the spliceosome and may also mediate mRNA trafficking in the axon and nerve terminus of neurons.

Antisense technology is an effective means for modulating the expression of one or more specific gene products, including alternative splice products, and is uniquely useful in a number of therapeutic, diagnostic, and research applications. The principle behind antisense technology is that an antisense compound, which hybridizes to a target nucleic acid, modulates gene expression activities such as transcription, splicing or translation through one of a number of antisense mechanisms. The sequence specificity of antisense compounds makes them extremely attractive as tools for target validation and gene functionalization, as well as therapeutics to selectively modulate the expression of genes involved in disease.

Certain antisense compounds complementary to SMN2 are known in the art. See for example, WO 2007/002390; U.S. 61/168,885; Hua et al., American J. of Human Genetics (April 2008) 82, 1-15; Singh et al., RNA Bio. 6:3, 1-10 (2009); WO2010120820 (2010). Chimeric peptide nucleic acid molecules designed to modulate splicing of SMN2 have been described (WO 02/38738; Cartegni and Krainer, Nat. Struct. Biol., 2003, 10, 120-125).

### SUMMARY OF THE INVENTION

In certain embodiments, the present invention provides methods of treating a subject having spinal muscular atrophy. In certain such embodiments, at least one GF/IGF-1 axis modulator is administered to the subject. In certain embodiments, at least one at least one GF/IGF-1 axis modulator and at least one antisense compound that modulates splicing of SMN2 to increase the amount of active exon 7 retained SMN protein is administered to the subject. In certain embodiments, at least one of the GF/IGF-1 axis modulator and the antisense compound is administered systemically. In certain embodiments, at least one of the GF/IGF-1 axis modulator and the antisense compound is administered into the CSF.

The present disclosure provides the following non-limiting numbered embodiments.

Embodiment 1: A method comprising administering at least one GF/IGF-1 axis modulator to a subject having spinal muscular atrophy (SMA).

Embodiment 2: The method of embodiment 1, wherein at least one GF/IGF-1 axis modulator is a GF/IGF-1 axis molecule.

Embodiment 3: The method of embodiment 1 or 2, wherein at least one GF/IGF-1 axis modulator increases the activity and/or amount of insulin-like growth factor 1 (IGF-1) in the subject.

Embodiment 4: The method of any of embodiments 1-3, wherein at least one GF/IGF-1 axis modulator is insulin-like growth factor 1 (IGF-1).

Embodiment 5: The method of any of embodiments 1-4, wherein at least one GF/IGF-1 axis modulator is IGF-binding-protein acid labile subunit (IGFALS).

Embodiment 6: The method of any of embodiments 1-5, wherein at least one GF/IGF-1 axis modulator is growth hormone (GH).

Embodiment 7: The method of any of embodiments 1-6, wherein at least one GF/IGF-1 axis modulator is insulin-like growth factor binding protein 3 (IGFBP3).

Embodiment 8: The method of any of embodiments 1-7 wherein at least one GF/IGF-1 axis modulator is a gene encoding a GF/IGF-1 axis molecule.

Embodiment 9: The method of any of embodiments 1-7 wherein at least one GF/IGF-1 axis modulator is a gene encoding IGF-binding-protein acid labile subunit (IGFALS).

Embodiment 10: The method of any embodiments 1-9, wherein at least one GF/IGF-1 axis modulator is administered systemically.

Embodiment 11: The method of any embodiments 1-10, wherein at least one growth hormone axis modulator is administered by intraperitoneal injection.

Embodiment 12: The method of any embodiments 1-11, wherein at least one growth hormone axis modulator is administered by subcutaneous injection.

Embodiment 13: The method of any embodiments 1-12, wherein at least one growth hormone axis modulator is administered by intramuscular injection.

Embodiment 14: The method of any embodiments 1-13, wherein at least one growth hormone axis modulator is administered into the cerebrospinal fluid.

Embodiment 15: The method of any of embodiments 1-14 comprising administering at least one antisense oligonucleotide to the subject having spinal muscular atrophy.

Embodiment 16: The method of embodiment 15, wherein the antisense compound comprises an antisense oligonucleotide complementary to a nucleic acid encoding human SMN2.

Embodiment 17: The method of embodiment 15 or 16, wherein the oligonucleotide is complementary to a portion of intron 7 of the nucleic acid encoding human SMN2.

Embodiment 18: The method of any of embodiments 15-17, wherein the antisense oligonucleotide is at least 90% complementary to the nucleic acid encoding human SMN2.

Embodiment 19: The method of embodiment 18, wherein the antisense oligonucleotide is fully complementary to the nucleic acid encoding human SMN2.

Embodiment 20: The method of any of embodiments 15-19, wherein the oligonucleotide has a nucleobase sequence comprising at least 10 contiguous nucleobases of the nucleobase sequence SEQ ID NO: 1.

Embodiment 21: The method of embodiment 20, wherein the oligonucleotide has a nucleobase sequence comprising at least 15 contiguous nucleobases of the nucleobase sequence SEQ ID NO: 1.

Embodiment 22: The method of embodiment 20, wherein the oligonucleotide has a nucleobase sequence comprising the nucleobase sequence SEQ ID NO: 1.

Embodiment 23: The method of embodiment 20, wherein the oligonucleotide has a nucleobase sequence consisting of the nucleobase sequence SEQ ID NO: 1.

Embodiment 24: The method of any of embodiments 15-23, wherein at least one nucleoside of the antisense oligonucleotide comprises a modified sugar moiety.

Embodiment 25: The method of embodiment 24, wherein the at least one modified sugar moiety comprises a 2′-methoxyethyl sugar moiety.

Embodiment 26: The method of any of embodiments 15-25, wherein essentially each nucleoside of the antisense oligonucleotide comprises a modified sugar moiety.

Embodiment 27: The method of embodiment 26, wherein the nucleosides comprising a modified sugar moiety all comprise the same sugar modification.

Embodiment 28: The method of embodiment 29, wherein each modified sugar moiety comprises a 2′-methoxyethyl sugar moiety.

Embodiment 29: The method of any of embodiments 15-28, wherein each nucleoside of the antisense oligonucleotide comprises a modified sugar moiety.

Embodiment 30: The method of embodiment 29, wherein the nucleosides all comprise the same sugar modification.

Embodiment 31: The method of embodiment 30, wherein each modified sugar moiety comprises a 2′-methoxyethyl sugar moiety.

Embodiment 32: The method of any of embodiments 15-31, wherein at least one modified nucleoside is a morpholino nucleoside.

Embodiment 33: The method of any of embodiments 15-32, wherein at least one modified nucleoside is a F-HNA nucleoside.

Embodiment 34: The method of any of embodiments 15-33, wherein at least one internucleoside linkage is a phosphorothioate internucleoside linkage.

Embodiment 35: The method of embodiment 34, wherein each internucleoside linkage is a phosphorothioate internucleoside linkage.

Embodiment 36: The method of any of embodiments 15-35, wherein the antisense oligonucleotide consists of 10 to 25 linked nucleosides.

Embodiment 37: The method of any of embodiments 15-35, wherein the antisense oligonucleotide consists of 12 to 22 linked nucleosides.

Embodiment 38: The method of any of embodiments 15-35, wherein the antisense oligonucleotide consists of 15 to 20 linked nucleosides.

Embodiment 39: The method of any of embodiments 15-35, wherein the antisense oligonucleotide consists of 18 linked nucleosides.

Embodiment 40: The method of any of embodiments 15-39, wherein the antisense compound comprises a conjugate group or terminal group.

Embodiment 41: The method of any of embodiments 15-40, wherein the antisense compound consists of the antisense oligonucleotide.

Embodiment 42: The method of any of embodiments 15-41, wherein the antisense compound is administered into the cerebrospinal fluid.

Embodiment 43: The method of embodiment 42, wherein the administration is into the intrathecal space.

Embodiment 44: The method of embodiment 42, wherein the administration is into the cerebrospinal fluid in the brain.

Embodiment 45: The method of any of embodiments 15-44, wherein the antisense compound is administered systemically.

Embodiment 46: Then method of embodiment 45, wherein the systemic administration of the antisense compound is by intravenous or intraperitoneal injection.

Embodiment 47: The method of embodiment 15-46, wherein the antisense compound is administered into the cerebrospinal fluid and by systemic administration at the same time.

Embodiment 48: The method of embodiment 15-47, wherein the antisense compound is administered into the cerebrospinal fluid and by systemic administration at different times.

Embodiment 49: The method of any of embodiments 1-48, wherein the administration of the GF/IGF-1 axis modulator and/or the antisense compound comprises a bolus injection.

Embodiment 50: The method of any of embodiments 1-49, wherein the administration of the GF/IGF-1 axis modulator and/or the antisense compound comprises infusion with a delivery pump.

Embodiment 51: The method of any of embodiments 15-50, wherein at least one GF/IGF-1 axis modulator and at least one antisense compound are administered at the same time.

Embodiment 52: The method of any of embodiments 15-51, wherein at least one GF/IGF-1 axis modulator and at least one antisense compound are administered at different times.

Embodiment 53: The method of any of embodiments 1-52, wherein the subject has type I SMA

Embodiment 54: The method of any of embodiments 1-52, wherein the subject has type II SMA.

Embodiment 55: The method of any of embodiments 1-52, embodiments wherein the subject has type III SMA.

Embodiment 56: The method of any of embodiments 1-55, wherein a first dose is administered in utero.

Embodiment 57: The method of any of embodiments 1-55, wherein a first dose of GF/IGF-1 axis modulator is administered within 1 week of birth of the subject.

Embodiment 58: The method of any of embodiments 1-55, wherein a first dose of GF/IGF-1 axis modulator is administered within 1 month of birth of the subject.

Embodiment 59: The method of any of embodiments 1-55, wherein a first dose of GF/IGF-1 axis modulator is administered within 3 months of birth of the subject.

Embodiment 60: The method of any of embodiments 1-55, wherein a first dose of GF/IGF-1 axis modulator is administered within 6 months of birth of the subject.

Embodiment 61: The method of any of embodiments 1-55, wherein a first dose of GF/IGF-1 axis modulator is administered when the subject is from 1 to 2 years of age.

Embodiment 62: The method of any of embodiments 1-55, wherein a first dose of GF/IGF-1 axis modulator is administered when the subject is from 1 to 15 years of age.

Embodiment 63: The method of any of embodiments 1-55, wherein a first dose of GF/IGF-1 axis modulator is administered when the subject is older than 15 years of age.

Embodiment 64: The method of any of embodiments 1-63, wherein the subject is a mammal.

Embodiment 65: The method of embodiment 64, wherein the subject is a human.

Embodiment 66: The method of any of embodiments 1-65 comprising identifying a subject having SMA.

Embodiment 67: The method of embodiment 66, wherein the subject is identified by measuring electrical activity of one or more muscles of the subject.

Embodiment 68: The method of embodiment 66 or 67, wherein the subject is identified by a genetic test to determine whether the subject has a mutation in the subject's SMN1 gene.

Embodiment 69: The method of any of embodiments 66-68, wherein the subject is identified by muscle biopsy.

Embodiment 70: The method of any of embodiments 1-69, wherein at least one symptom of SMA in the subject is ameliorated.

In certain embodiments, the subject is treated with gene therapy. In certain embodiments, the gene therapy is in the CSF. In certain embodiments, gene therapy is systemic. In certain embodiments, the gene therapy provides exon-7 retained SMN. In certain embodiments, the gene therapy increases the GH/IGF-1 axis.

### BRIEF DESCRIPTION OF THE FIGURES

FIG. 1 shows results from Experiments described in Examples 3 and 4. FIG. 1a shows Serum IGF-1 from heterozygous (normal phenotype) mice, SMA mice, and SMA mice treated systemically with antisense compound. FIGS. 1b-d show RT-PCR results from experiments described in Example 4 in which liver RNA was assessed for mRNA encoding IGF-1, IGFBP3 and IGFALS.

### DETAILED DESCRIPTION OF THE INVENTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose.

### Definitions

Unless specific definitions are provided, the nomenclature used in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 21^{st} edition, 2005; and “Antisense Drug Technology, Principles, Strategies, and Applications” Edited by Stanley T. Crooke, CRC Press, Boca Raton, Fla.; and Sambrook et al., “Molecular Cloning, A laboratory Manual,” 2^{nd} Edition, Cold Spring Harbor Laboratory Press, 1989, which are hereby incorporated by reference for any purpose. Where permitted, all patents, applications, published applications and other publications and other data referred to throughout in the disclosure are incorporated by reference herein in their entirety.

Unless otherwise indicated, the following terms have the following meanings:

As used herein, “nucleoside” means a compound comprising a nucleobase moiety and a sugar moiety. Nucleosides include, but are not limited to, naturally occurring nucleosides (as found in DNA and RNA) and modified nucleosides. Nucleosides may be linked to a phosphate moiety.

As used herein, “chemical modification” means a chemical difference in a compound when compared to a naturally occurring counterpart. In reference to an oligonucleotide, chemical modification does not include differences only in nucleobase sequence. Chemical modifications of oligonucleotides include nucleoside modifications (including sugar moiety modifications and nucleobase modifications) and internucleoside linkage modifications.

As used herein, “furanosyl” means a structure comprising a 5-membered ring comprising four carbon atoms and one oxygen atom.

As used herein, “naturally occurring sugar moiety” means a ribofuranosyl as found in naturally occurring RNA or a deoxyribofuranosyl as found in naturally occurring DNA.

As used herein, “sugar moiety” means a naturally occurring sugar moiety or a modified sugar moiety of a nucleoside.

As used herein, “modified sugar moiety” means a substituted sugar moiety, a bicyclic or tricyclic sugar moiety, or a sugar surrogate.

As used herein, “substituted sugar moiety” means a furanosyl comprising at least one substituent group that differs from that of a naturally occurring sugar moiety. Substituted sugar moieties include, but are not limited to furanosyls comprising substituents at the 2′-position, the 3′-position, the 5′-position and/or the 4′-position.

As used herein, “2′-substituted sugar moiety” means a furanosyl comprising a substituent at the 2′-position other than H or OH. Unless otherwise indicated, a 2′-substituted sugar moiety is not a bicyclic sugar moiety (i.e., the 2′-substituent of a 2′-substituted sugar moiety does not form a bridge to another atom of the furanosyl ring.

As used herein, “MOE” means —OCH₂CH₂OCH₃.

As used herein, “bicyclic sugar moiety” means a modified sugar moiety comprising a 4 to 7 membered ring (including but not limited to a furanosyl) comprising a bridge connecting two atoms of the 4 to 7 membered ring to form a second ring, resulting in a bicyclic structure. In certain embodiments, the 4 to 7 membered ring is a sugar ring. In certain embodiments the 4 to 7 membered ring is a furanosyl. In certain such embodiments, the bridge connects the 2′-carbon and the 4′-carbon of the furanosyl.

As used herein the term “sugar surrogate” means a structure that does not comprise a furanosyl and that is capable of replacing the naturally occurring sugar moiety of a nucleoside, such that the resulting nucleoside is capable of (1) incorporation into an oligonucleotide and (2) hybridization to a complementary nucleoside. Such structures include rings comprising a different number of atoms than furanosyl (e.g., 4, 6, or 7-membered rings); replacement of the oxygen of a furanosyl with a non-oxygen atom (e.g., carbon, sulfur, or nitrogen); or both a change in the number of atoms and a replacement of the oxygen. Such structures may also comprise substitutions corresponding to those described for substituted sugar moieties (e.g., 6-membered carbocyclic bicyclic sugar surrogates optionally comprising additional substituents). Sugar surrogates also include more complex sugar replacements (e.g., the non-ring systems of peptide nucleic acid). Sugar surrogates include without limitation morpholino, modified morpholinos, cyclohexenyls and cyclohexitols.

As used herein, “nucleotide” means a nucleoside further comprising a phosphate linking group. As used herein, “linked nucleosides” may or may not be linked by phosphate linkages and thus includes, but is not limited to “linked nucleotides.” As used herein, “linked nucleosides” are nucleosides that are connected in a continuous sequence (i.e. no additional nucleosides are present between those that are linked).

As used herein, “nucleobase” means a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Nucleobases may be naturally occurring or may be modified.

As used herein, “heterocyclic base” or “heterocyclic nucleobase” means a nucleobase comprising a heterocyclic structure.

As used herein the terms, “unmodified nucleobase” or “naturally occurring nucleobase” means the naturally occurring heterocyclic nucleobases of RNA or DNA: the purine bases adenine (A) and guanine (G), and the pyrimidine bases thymine (T), cytosine (C) (including 5-methyl C), and uracil (U).

As used herein, “modified nucleobase” means any nucleobase that is not a naturally occurring nucleobase.

As used herein, “modified nucleoside” means a nucleoside comprising at least one chemical modification compared to naturally occurring RNA or DNA nucleosides. Modified nucleosides comprise a modified sugar moiety and/or a modified nucleobase.

As used herein, “bicyclic nucleoside” or “BNA” means a nucleoside comprising a bicyclic sugar moiety.

As used herein, “constrained ethyl nucleoside” or “cEt” means a nucleoside comprising a bicyclic sugar moiety comprising a 4′—CH(CH₃)—O-2′bridge.

As used herein, “locked nucleic acid nucleoside” or “LNA” means a nucleoside comprising a bicyclic sugar moiety comprising a 4′—CH₂—O-2′bridge.

As used herein, “2′-substituted nucleoside” means a nucleoside comprising a substituent at the 2′-position other than H or OH. Unless otherwise indicated, a 2′-substituted nucleoside is not a bicyclic nucleoside.

As used herein, “2′-deoxynucleoside” means a nucleoside comprising 2′-H furanosyl sugar moiety, as found in naturally occurring deoxyribonucleosides (DNA). In certain embodiments, a 2′-deoxynucleoside may comprise a modified nucleobase or may comprise an RNA nucleobase (e.g., uracil).

As used herein, “oligonucleotide” means a compound comprising a plurality of linked nucleosides. In certain embodiments, an oligonucleotide comprises one or more unmodified ribonucleosides (RNA) and/or unmodified deoxyribonucleosides (DNA) and/or one or more modified nucleosides.

As used herein “oligonucleoside” means an oligonucleotide in which none of the internucleoside linkages contains a phosphorus atom. As used herein, oligonucleotides include oligonucleosides.

As used herein, “modified oligonucleotide” means an oligonucleotide comprising at least one modified nucleoside and/or at least one modified internucleoside linkage.

As used herein “internucleoside linkage” means a covalent linkage between adjacent nucleosides in an oligonucleotide.

As used herein “naturally occurring internucleoside linkage” means a 3′ to 5′ phosphodiester linkage.

As used herein, “modified internucleoside linkage” means any internucleoside linkage other than a naturally occurring internucleoside linkage.

As used herein, “oligomeric compound” means a polymeric structure comprising two or more sub-structures. In certain embodiments, an oligomeric compound comprises an oligonucleotide. In certain embodiments, an oligomeric compound comprises one or more conjugate groups and/or terminal groups. In certain embodiments, an oligomeric compound consists of an oligonucleotide.

As used herein, “terminal group” means one or more atom attached to either, or both, the 3′ end or the 5′ end of an oligonucleotide. In certain embodiments a terminal group is a conjugate group. In certain embodiments, a terminal group comprises one or more terminal group nucleosides.

As used herein, “conjugate” means an atom or group of atoms bound to an oligonucleotide or oligomeric compound. In general, conjugate groups modify one or more properties of the compound to which they are attached, including, but not limited to pharmacodynamic, pharmacokinetic, binding, absorption, cellular distribution, cellular uptake, charge and/or clearance properties.

As used herein, “conjugate linking group” means any atom or group of atoms used to attach a conjugate to an oligonucleotide or oligomeric compound.

As used herein, “antisense compound” means a compound comprising or consisting of an oligonucleotide at least a portion of which is complementary to a target nucleic acid to which it is capable of hybridizing, resulting in at least one antisense activity.

As used herein, “antisense activity” means any detectable and/or measurable change attributable to the hybridization of an antisense compound to its target nucleic acid.

As used herein, “detecting” or “measuring” means that a test or assay for detecting or measuring is performed. Such detection and/or measuring may result in a value of zero. Thus, if a test for detection or measuring results in a finding of no activity (activity of zero), the step of detecting or measuring the activity has nevertheless been performed.

As used herein, “detectable and/or measurable activity” means a statistically significant activity that is not zero.

As used herein, “essentially unchanged” means little or no change in a particular parameter, particularly relative to another parameter which changes much more. In certain embodiments, a parameter is essentially unchanged when it changes less than 5%. In certain embodiments, a parameter is essentially unchanged if it changes less than two-fold while another parameter changes at least ten-fold. For example, in certain embodiments, an antisense activity is a change in the amount of a target nucleic acid. In certain such embodiments, the amount of a non-target nucleic acid is essentially unchanged if it changes much less than the target nucleic acid does, but the change need not be zero.

As used herein, “expression” means the process by which a gene ultimately results in a protein. Expression includes, but is not limited to, transcription, post-transcriptional modification (e.g., splicing, polyadenlyation, addition of 5′-cap), and translation.

As used herein, “target nucleic acid” means a nucleic acid molecule to which an antisense compound hybridizes.

As used herein, “mRNA” means an RNA molecule that encodes a protein.

As used herein, “pre-mRNA” means an RNA transcript that has not been fully processed into mRNA. Pre-RNA includes one or more intron.

As used herein, “transcript” means an RNA molecule transcribed from DNA. Transcripts include, but are not limited to non-coding RNA, mRNA, pre-mRNA, and partially processed RNA.

As used herein, “targeting” or “targeted to” means the association of an antisense compound to a particular target nucleic acid molecule or a particular region of a target nucleic acid molecule. An antisense compound targets a target nucleic acid if it is sufficiently complementary to the target nucleic acid to allow hybridization under physiological conditions.

As used herein, “nucleobase sequence” means the order of contiguous nucleobases, independent of any sugar, linkage, and/or nucleobase modification.

As used herein, “nucleobase complementarity” or “complementarity” when in reference to nucleobases means a nucleobase that is capable of base pairing with another nucleobase. For example, in DNA, adenine (A) is complementary to thymine (T). For example, in RNA, adenine (A) is complementary to uracil (U). In certain embodiments, complementary nucleobase means a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair. Nucleobases comprising certain modifications may maintain the ability to pair with a counterpart nucleobase and thus, are still capable of nucleobase complementarity.

As used herein, “non-complementary” in reference to nucleobases means a pair of nucleobases that do not form hydrogen bonds with one another.

As used herein, “complementary” in reference to oligomeric compounds (e.g., linked nucleosides, oligonucleotides, or nucleic acids) means the capacity of such oligomeric compounds or regions thereof to hybridize to another oligomeric compound or region thereof through nucleobase complementarity under stringent conditions. Complementary oligomeric compounds need not have nucleobase complementarity at each nucleoside. Rather, some mismatches are tolerated. In certain embodiments, complementary oligomeric compounds or regions are complementary at 70% of the nucleobases (70% complementary). In certain embodiments, complementary oligomeric compounds or regions are 80% complementary. In certain embodiments, complementary oligomeric compounds or regions are 90% complementary. In certain embodiments, complementary oligomeric compounds or regions are 95% complementary. In certain embodiments, complementary oligomeric compounds or regions are 100% complementary.

As used herein, “hybridization” means the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleobases.

As used herein, “specifically hybridizes” means the ability of an oligomeric compound to hybridize to one nucleic acid site with greater affinity than it hybridizes to another nucleic acid site. In certain embodiments, an antisense oligonucleotide specifically hybridizes to more than one target site.

As used herein, “percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound.

As used herein, “percent identity” means the number of nucleobases in a first nucleic acid that are the same type (independent of chemical modification) as nucleobases at corresponding positions in a second nucleic acid, divided by the total number of nucleobases in the first nucleic acid.

As used herein, “modulation” means a change of amount or quality of a molecule, function, or activity when compared to the amount or quality of a molecule, function, or activity prior to modulation. For example, modulation includes the change, either an increase (stimulation or induction) or a decrease (inhibition or reduction) in gene expression. As a further example, modulation of expression can include a change in splice site selection of pre-mRNA processing, resulting in a change in the absolute or relative amount of a particular splice-variant compared to the amount in the absence of modulation.

As used herein, “motif” means a pattern of chemical modifications in an oligomeric compound or a region thereof. Motifs may be defined by modifications at certain nucleosides and/or at certain linking groups of an oligomeric compound.

As used herein, “nucleoside motif” means a pattern of nucleoside modifications in an oligomeric compound or a region thereof. The linkages of such an oligomeric compound may be modified or unmodified. Unless otherwise indicated, motifs herein describing only nucleosides are intended to be nucleoside motifs. Thus, in such instances, the linkages are not limited.

As used herein, “sugar motif” means a pattern of sugar modifications in an oligomeric compound or a region thereof.

As used herein, “linkage motif” means a pattern of linkage modifications in an oligomeric compound or region thereof. The nucleosides of such an oligomeric compound may be modified or unmodified. Unless otherwise indicated, motifs herein describing only linkages are intended to be linkage motifs. Thus, in such instances, the nucleosides are not limited.

As used herein, “nucleobase modification motif” means a pattern of modifications to nucleobases along an oligonucleotide. Unless otherwise indicated, a nucleobase modification motif is independent of the nucleobase sequence.

As used herein, “sequence motif” means a pattern of nucleobases arranged along an oligonucleotide or portion thereof. Unless otherwise indicated, a sequence motif is independent of chemical modifications and thus may have any combination of chemical modifications, including no chemical modifications.

As used herein, “fully modified motif” means an oligonucleotide or a portion thereof wherein each nucleobase, each sugar, and/or each internucleoside linkage is modified.

As used herein, “uniformly modified motif” means an oligonucleotide or a portion thereof wherein each nucleobase, each sugar, and/or each internucleoside linkage has the same modification throughout the modified oligonucleotide or portion thereof.

As used herein, “alternating motif” means an oligonucleotide or a portion thereof, having at least four separate regions of modified nucleosides in a pattern (AB)ₙAₘ where A represents a region of nucleosides having a first type of modification; B represent a region of nucleosides having a different type of modification; n is 2-15; and m is 0 or 1. Thus, in certain embodiments, alternating motifs include 4, 5, 6, 7, 8, 9, 10, 11, 12, 13, 14, 15, 16, 17, 18, 19, or 20 or more alternating regions. In certain embodiments, each A region and each B region independently comprises 1-4 nucleosides.

As used herein, “type of modification” in reference to a nucleoside or a nucleoside of a “type” means the chemical modification of a nucleoside and includes modified and unmodified nucleosides. Accordingly, unless otherwise indicated, a “nucleoside having a modification of a first type” may be an unmodified nucleoside.

As used herein, “differently modified” mean chemical modifications or chemical substituents that are different from one another, including absence of modifications. Thus, for example, a MOE nucleoside and an unmodified DNA nucleoside are “differently modified,” even though the DNA nucleoside is unmodified. Likewise, DNA and RNA are “differently modified,” even though both are naturally-occurring unmodified nucleosides. Nucleosides that are the same but for comprising different nucleobases are not differently modified. For example, a nucleoside comprising a 2′-OMe modified sugar and an unmodified adenine nucleobase and a nucleoside comprising a 2′-OMe modified sugar and an unmodified thymine nucleobase are not differently modified.

As used herein, “the same type of modifications” refers to modifications that are the same as one another, including absence of modifications. Thus, for example, two unmodified DNA nucleoside have “the same type of modification,” even though the DNA nucleoside is unmodified. Such nucleosides having the same type modification may comprise different nucleobases.

As used herein, “pharmaceutically acceptable carrier or diluent” means any substance suitable for use in administering to an animal. In certain embodiments, a pharmaceutically acceptable carrier or diluent is sterile saline. In certain embodiments, such sterile saline is pharmaceutical grade saline.

As used herein, “animal” includes human and non-human animals.

As used herein, “subject” means a human or non-human animal selected for treatment or therapy.

As used herein, “subject in need thereof” means a subject identified as in need of a therapy or treatment. In such embodiments, a subject has one or more indications of having or developing SMA.

As used herein, “administering” means providing a pharmaceutical agent or composition to a subject, and includes, but is not limited to, administering by a medical professional and self-administering.

As used herein, “parenteral administration,” means administration through injection or infusion. Parenteral administration includes, but is not limited to, subcutaneous administration, intravenous administration, or intramuscular administration.

As used herein, “systemic administration” means administration to an area other than the intended locus of activity. Examples or systemic administration are subcutaneous administration and intravenous administration, and intraperitoneal administration.

As used herein, “subcutaneous administration” means administration just below the skin.

As used herein, “intravenous administration” means administration into a vein.

As used herein, “cerebrospinal fluid” or “CSF” means the fluid filling the space around the brain and spinal cord.

As used herein, “administration into the cerebrospinal fluid” means any administration that delivers a substance directly into the CSF.

As used herein, “intracerebroventricular” or “ICV” mean administration into the ventricular system of the brain.

As used herein, “intrathecal” or “IT” means administration into the CSF under the arachnoid membrane which covers the brain and spinal cord. IT injection is performed through the theca of the spinal cord into the subarachnoid space, where a pharmaceutical agent is injected into the sheath surrounding the spinal cord.

As used herein, “amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

As used herein, “prevent the onset of” means to prevent the development a condition or disease in a subject who is at risk for developing the disease or condition. In certain embodiments, a subject at risk for developing the disease or condition receives treatment similar to the treatment received by a subject who already has the disease or condition.

As used herein, “delay the onset of” means to delay the development of a condition or disease in a subject who is at risk for developing the disease or condition.

As used herein, “slow the progression of” means that the severity of at least one symptom associated with a disease or condition worsens less quickly. As used herein, “exon 7 amino acids” means the portion of an SMN protein that correspond to exon 7 of the SMN RNA. Exon 7 amino acids are present in SMN protein expressed from SMN RNA where exon 7 was not excluded during splicing.

As used herein, “SMN protein” means normal full length survival motor neuron protein. SMN may be expressed from either an SMN1 gene or from an SMN2 gene, provided that exon 7 is present in the mature mRNA and the exon 7 amino acids are present in the SMN protein.

As used herein, “dose” means a specified quantity of a pharmaceutical agent provided in a single administration or over a specified amount of time. In certain embodiments, a dose may be administered in two or more boluses, tablets, or injections. For example, in certain embodiments, where subcutaneous or inrathecal or ICV administration is desired, the desired dose requires a volume not easily accommodated by a single injection. In such embodiments, two or more injections may be used to achieve the desired dose. In the setting of continuous infusion, dose may be expressed as the quantity of a pharmaceutical agent delivered per unit of time.

As used herein, “therapeutically effective amount” means an amount of a pharmaceutical agent that provides a therapeutic benefit to an animal.

As used herein, “growth hormone” or “GH” means a hormone secreted by somatotropic cells under control of hypothalamic factor in an animal and analogs thereof, whether produced naturally in an animal, in cells or extracts, or chemically synthesized.

As used herein, “growth hormone/insulin-like growth factor 1 axis” or “GF/IGF-1 axis” means a system of biological molecules (“GF/IGF-1 axis molecules”) that include growth hormone and insulin-like growth factor 1 (IGF-1) and moleculest that modulate and/or are modulated by growth hormone and or insulin-like growth factor 1 (IGF-1). GF/IGF-1 axis molecules include but are not limited to: growth hormone, insulin-like growth factor 1 (IGF-1), insulin-like growth factor 2 (IGF-2); insulin-like growth factor 1 receptor (IGFR-1), insulin-like growth factor 2 receptor (IGFR-2), insulin-like growth factor binding protein 1 (IGBP-1), insulin-like growth factor binding protein 2 (IGBP-2), insulin-like growth factor binding protein 3 (IGBP-3), insulin-like growth factor binding protein 4 (IGBP-4), insulin-like growth factor binding protein 5 (IGBP-5), insulin-like growth factor binding protein 6 (IGBP-6), IGF degrading proteins, and insulin-like growth factor binding protein acid labile subunit (IGFALS).

As used herein, “GF/IGF-1 axis modulator” refers a molecule that modulates the GF/IGF-1 axis in an animal other than an antisense compound that alters splicing of SMN2. GF/IGF-1 axis modulators include, but are not limited to natural molecules that make up the GF/IGF-1 axis (GF/IGF-1 axis molecules) and analogs thereof, whether produced in cells or chemically synthesized, as well as other chemical or biologic molecules that modulate the amount or activity of such GF/IGF-1 axis molecules. GF/IGF-1 axis modulators typically affect the amount, activity, sensitivity, and/or stability of one or more GF/IGF-1 axis molecule. In certain embodiments, a GF/IGF-1 axis modulator affects the stability of one or more GF/IGF-1 axis molecule. In certain embodiments, a GF/IGF-1 axis modulator affects the distribution or localization of one or more GF/IGF-1 axis molecule in a subject.

As used herein, “substituent” and “substituent group,” means an atom or group that replaces the atom or group of a named parent compound. For example a substituent of a modified nucleoside is any atom or group that differs from the atom or group found in a naturally occurring nucleoside (e.g., a modified 2′-substuent is any atom or group at the 2′-position of a nucleoside other than H or OH). Substituent groups can be protected or unprotected. In certain embodiments, compounds of the present invention have substituents at one or at more than one position of the parent compound. Substituents may also be further substituted with other substituent groups and may be attached directly or via a linking group such as an alkyl or hydrocarbyl group to a parent compound.

Likewise, as used herein, “substituent” in reference to a chemical functional group means an atom or group of atoms differs from the atom or a group of atoms normally present in the named functional group. In certain embodiments, a substituent replaces a hydrogen atom of the functional group (e.g., in certain embodiments, the substituent of a substituted methyl group is an atom or group other than hydrogen which replaces one of the hydrogen atoms of an unsubstituted methyl group). Unless otherwise indicated, groups amenable for use as substituents include without limitation, halogen, hydroxyl, alkyl, alkenyl, alkynyl, acyl (—C(O)Rₐₐ), carboxyl (—C(O)O—Rₐₐ), aliphatic groups, alicyclic groups, alkoxy, substituted oxy (—O—Rₐₐ), aryl, aralkyl, heterocyclic radical, heteroaryl, heteroarylalkyl, amino (—N(R_{bb})(R_{cc})), imino(═NR_{bb}), amido (—C(O)N(R_{bb})(R_{cc}) or —N(R_{bb})C(O)Rₐₐ), azido (—N₃), nitro (—NO₂), cyano (—CN), carbamido (—OC(O)N(R_{bb})(R_{cc}) or —N(R_{bb})C(O)N(Rₐₐ), ureido (—N(R_{bb})C(O)N(R_{bb})(R_{cc})), thioureido (—N(R_{bb})C(S)N(R_{bb})—(R_{cc})), guanidinyl (—N(R_{bb})C(═NR_{bb})N(R_{bb})(R_{cc})), amidinyl (—C(═NR_{bb})N(R_{bb})(R_{cc}) or —N(R_{bb})C(═NR_{bb})(Rₐₐ)), thiol (—SR_{bb}), sulfinyl (—S(O)R_{bb}), sulfonyl (—S(O)₂R_{bb}) and sulfonamidyl (—S(O)₂N(R_{bb})(R_{cc}) or —N(R_{bb})S—(O)₂R_{bb}). Wherein each Rₐₐ, R_{bb} and R_{cc} is, independently, H, an optionally linked chemical functional group or a further substituent group with a preferred list including without limitation, alkyl, alkenyl, alkynyl, aliphatic, alkoxy, acyl, aryl, aralkyl, heteroaryl, alicyclic, heterocyclic and heteroarylalkyl. Selected substituents within the compounds described herein are present to a recursive degree.

As used herein, “alkyl,” as used herein, means a saturated straight or branched hydrocarbon radical containing up to twenty four carbon atoms. Examples of alkyl groups include without limitation, methyl, ethyl, propyl, butyl, isopropyl, n-hexyl, octyl, decyl, dodecyl and the like. Alkyl groups typically include from 1 to about 24 carbon atoms, more typically from 1 to about 12 carbon atoms (C₁-C₁₂ alkyl) with from 1 to about 6 carbon atoms being more preferred.

As used herein, “alkenyl,” means a straight or branched hydrocarbon chain radical containing up to twenty four carbon atoms and having at least one carbon-carbon double bond. Examples of alkenyl groups include without limitation, ethenyl, propenyl, butenyl, 1-methyl-2-buten-1-yl, dienes such as 1,3-butadiene and the like. Alkenyl groups typically include from 2 to about 24 carbon atoms, more typically from 2 to about 12 carbon atoms with from 2 to about 6 carbon atoms being more preferred. Alkenyl groups as used herein may optionally include one or more further substituent groups.

As used herein, “alkynyl,” means a straight or branched hydrocarbon radical containing up to twenty four carbon atoms and having at least one carbon-carbon triple bond. Examples of alkynyl groups include, without limitation, ethynyl, 1-propynyl, 1-butynyl, and the like. Alkynyl groups typically include from 2 to about 24 carbon atoms, more typically from 2 to about 12 carbon atoms with from 2 to about 6 carbon atoms being more preferred. Alkynyl groups as used herein may optionally include one or more further substituent groups.

As used herein, “acyl,” means a radical formed by removal of a hydroxyl group from an organic acid and has the general Formula —C(O)—X where X is typically aliphatic, alicyclic or aromatic. Examples include aliphatic carbonyls, aromatic carbonyls, aliphatic sulfonyls, aromatic sulfinyls, aliphatic sulfinyls, aromatic phosphates, aliphatic phosphates and the like. Acyl groups as used herein may optionally include further substituent groups.

As used herein, “alicyclic” means a cyclic ring system wherein the ring is aliphatic. The ring system can comprise one or more rings wherein at least one ring is aliphatic. Preferred alicyclics include rings having from about 5 to about 9 carbon atoms in the ring. Alicyclic as used herein may optionally include further substituent groups.

As used herein, “aliphatic” means a straight or branched hydrocarbon radical containing up to twenty four carbon atoms wherein the saturation between any two carbon atoms is a single, double or triple bond. An aliphatic group preferably contains from 1 to about 24 carbon atoms, more typically from 1 to about 12 carbon atoms with from 1 to about 6 carbon atoms being more preferred. The straight or branched chain of an aliphatic group may be interrupted with one or more heteroatoms that include nitrogen, oxygen, sulfur and phosphorus. Such aliphatic groups interrupted by heteroatoms include without limitation, polyalkoxys, such as polyalkylene glycols, polyamines, and polyimines. Aliphatic groups as used herein may optionally include further substituent groups.

As used herein, “alkoxy” means a radical formed between an alkyl group and an oxygen atom wherein the oxygen atom is used to attach the alkoxy group to a parent molecule. Examples of alkoxy groups include without limitation, methoxy, ethoxy, propoxy, isopropoxy, n-butoxy, sec-butoxy, tert-butoxy, n-pentoxy, neopentoxy, n-hexoxy and the like. Alkoxy groups as used herein may optionally include further substituent groups.

As used herein, “aminoalkyl” means an amino substituted C₁-C₁₂ alkyl radical. The alkyl portion of the radical forms a covalent bond with a parent molecule. The amino group can be located at any position and the aminoalkyl group can be substituted with a further substituent group at the alkyl and/or amino portions.

As used herein, “aralkyl” and “arylalkyl” mean an aromatic group that is covalently linked to a C₁-C₁₂ alkyl radical. The alkyl radical portion of the resulting aralkyl (or arylalkyl) group forms a covalent bond with a parent molecule. Examples include without limitation, benzyl, phenethyl and the like. Aralkyl groups as used herein may optionally include further substituent groups attached to the alkyl, the aryl or both groups that form the radical group.

As used herein, “aryl” and “aromatic” mean a mono- or polycyclic carbocyclic ring system radicals having one or more aromatic rings. Examples of aryl groups include without limitation, phenyl, naphthyl, tetrahydronaphthyl, indanyl, idenyl and the like. Preferred aryl ring systems have from about 5 to about 20 carbon atoms in one or more rings. Aryl groups as used herein may optionally include further substituent groups.

As used herein, “halo” and “halogen,” mean an atom selected from fluorine, chlorine, bromine and iodine.

As used herein, “heteroaryl,” and “heteroaromatic,” mean a radical comprising a mono- or poly-cyclic aromatic ring, ring system or fused ring system wherein at least one of the rings is aromatic and includes one or more heteroatoms. Heteroaryl is also meant to include fused ring systems including systems where one or more of the fused rings contain no heteroatoms. Heteroaryl groups typically include one ring atom selected from sulfur, nitrogen or oxygen. Examples of heteroaryl groups include without limitation, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl and the like. Heteroaryl radicals can be attached to a parent molecule directly or through a linking moiety such as an aliphatic group or hetero atom. Heteroaryl groups as used herein may optionally include further substituent groups.

### Spinal Muscular Atrophy (SMA)

SMA is a genetic disorder characterized by degeneration of spinal motor neurons. SMA is caused by the homozygous loss of both functional copies of the SMN1 gene. However, the SMN2 gene has the potential to code for the same protein as SMN1 and thus overcome the genetic defect of SMA patients. SMN2 contains a translationally silent mutation (C→T) at position +6 of exon 7, which results in inefficient inclusion of exon 7 in SMN2 transcripts. Therefore, the predominant form of SMN2 protein, one which lacks exon 7 amino acids, is unstable and inactive. Therapeutic compounds capable of modulating SMN2 splicing such that the percentage of SMN2 transcripts containing exon 7 is increased, have been shown in animal models to be useful for the treatment of SMA. See, e.g., WO 2010/148249, incorporated herein for any purpose. For example, an antisense compound complementary to a portion of intron 7 alters splicing of SMN2 to include exon 7, resulting in functional SMN protein. Dramatic therapeutic benefit was observed when such antisense compound was administered to the CSF of SMA mice.

Further, and surprisingly, systemic administration of such compounds was also shown to provide therapeutic benefit, either alone or in combination with administration to the CSF. Since oligonucleotides do not cross the blood-brain barrier, such systemic administration is expected result in reduced antisense compound in neurons. While some evidence suggests that functional SMN inside neurons is required for normal neuron function, the consequence of reduced functional SMN in other cells and tissues has not been well characterized.

### The GH/IGF-1 Axis

Insulin-like growth factor-1 (IGF-1) is secreted principally by the liver in response to growth hormone (GH). These molecules (GH and IGF-1) and molecules involved in regulation of these molecules are referred to as the GH/IGF-1 axis.

In certain instances, animals that have SMA, including SMA mice, are smaller than animals that do not have SMA. Thus, it was speculated that at least some of the observed therapeutic benefit of systemic administration of antisense compounds that alter splicing of SMN2 was attributable to the effect of reduced functional SMN protein on the GH/IGF-1 axis.

Certain embodiments of the present invention flow from the discovery that serum from SMA mice showed reduced IGF-1 compared to normal mice or to SMA mice treated systemically with an antisense compound the alters splicing of SMN2 to include exon 7. RT-PCR from such mice showed that hepatic mRNA of IGF-1 was not reduced, suggesting that the expression levels of IGF-1 are not altered. However, mRNA of IGF-binding-protein acid labile subunit (IGFALS) was reduced in SMA mice. IGFALS binds to IGF-1 and IGF-1 binding protein 3 (IGFBP3) forming a stable complex which extends the half life of IGF-1 from about 10 minutes to 12-15 hours. Thus, in certain circumstances, reduced functional SMN results in reduced IGFALS, which in turn results in decreased stability of IGF-1 (because it is not complexed with IGFALS) and ultimately reduced IGF-1 levels. In certain embodiments, reduced IGF-1 has physiologic consequences, including but not limited to cardiac defects, and poor growth.

IGF-1 is also a potent neurotrophic factor. It enhances growth-cone mobility, potentiates long-term neurite outgrowth, inhibits neuronal apoptosis, and promotes neurogenesis and synaptogenisis. Circulating IGF-1 crosses the blood-brain barrier. Thus, reduced circulating IGF-1 levels in the setting of SMA likely results in reduced IGF-1 in the CNS. Such reduced IGF-1 in the CNS may contribute to SMA neuropathology. Thus, in certain embodiments, restoration of normal HG/IGF-1 axis ameliorates symptoms in the CNS as well as symptoms outside the CNS.

In certain embodiments, the present invention provides methods of modulating the GH/IGF-1 axis in an animal having SMA. In certain embodiments, the present invention provides methods of increasing activity of the GH/IGF-1 axis in an animal having SMA. In certain embodiments, the invention provides methods of administering at least one GH/IGF-1 axis molecule (e.g., GH, IGF-1 or IGFALS). In certain embodiments, the invention provides administration of at least one compound that modulates the expression, activity and/or stability of at least one GH/IGF-1 axis molecule. In certain embodiments, the invention provides administration of at least one compound that increases the expression, activity and/or stability of at least one GH/IGF-1 axis molecule. In certain embodiments, the invention provides administration of at least one compound that increases activity of the GH/IGF-1 axis and one compound that alters splicing of SMN2 to increase inclusion of exon 7 in an animal having SMA.

In certain embodiments the invention provides administration of at least one GH/IGF-1 axis modulator to a subject with SMA. In certain embodiments, the GH/IGF-1 axis modulator is a gene encoding a GH/IGF-1 axis molecule. In certain embodiment, the GH/IGF-1 axis modulator is a GH/IGF-1 axis molecule. In certain embodiments, the GH/IGF-1 axis modulator is a compound that increases stability or expression of at least one GH/IGF-1 axis molecule.

In certain embodiments, an antisense compound that modulates splicing of SMN2 to increase the amount of exon 7 retained SMN protein is also administered.

### Oligomeric Compounds

In certain embodiments, the present invention provides oligomeric compounds. In certain embodiments, such oligomeric compounds comprise oligonucleotides optionally comprising one or more conjugate groups. In certain embodiments, an oligomeric compound consists of an oligonucleotide. In certain embodiments, the oligonucleotides comprise one or more chemical modifications. Such chemical modifications include modifications one or more nucleoside (including modifications to the sugar moiety and/or the nucleobase) and/or modifications to one or more internucleoside linkage.

### Certain Sugar Moieties

In certain embodiments, oligomeric compounds of the invention comprise one or more modifed nucleosides comprising a modifed sugar moiety. Such oligomeric compounds comprising one or more sugar-modified nucleosides may have desirable properties, such as enhanced nuclease stability or increased binding affinity with a target nucleic acid relative to oligomeric compounds comprising only nucleosides comprising naturally occurring sugar moieties. In certain embodiments, modified sugar moieties are substituted sugar moieties. In certain embodiments, modified sugar moieties are bicyclic or tricyclic sugar moieties. In certain embodiments, modified sugar moieties are sugar surrogates. Such sugar surogates may comprise one or more substitutions corresponding to those of substituted sugar moieties.

In certain embodiments, modified sugar moieties are substituted sugar moieties comprising one or more substituent, including but not limited to substituents at the 2′ and/or 5′ positions. Examples of sugar substituents suitable for the 2′-position, include, but are not limited to: 2′-F, 2′-OCH3(“OMe” or “O-methyl”), and 2′-O(CH₂)₂OCH₃(“MOE”). In certain embodiments, sugar substituents at the 2′ position is selected from allyl, amino, azido, thio, O-allyl, O—C₁-C₁₀ alkyl, O—C₁-C₁₀ substituted alkyl; O—C₁-C₁₀ alkoxy; O—C₁-C₁₀ substituted alkoxy, OCF₃, O(CH₂)₂SCH₃, O(CH₂)₂—O—N(Rm)(Rn), and O—CH₂—C(═O)—N(Rm)(Rn), where each Rm and Rn is, independently, H or substituted or unsubstituted C₁-C₁₀ alkyl. Examples of sugar substituents at the 5′-position, include, but are not limited to: 5′-methyl (R or S); 5′-vinyl, and 5′-methoxy. In certain embodiments, substituted sugars comprise more than one non-bridging sugar substituent, for example, 2′-F-5′-methyl sugar moieties (see, e.g., PCT International Application WO 2008/101157, for additional 5′, 2′-bis substituted sugar moieties and nucleosides).

Nucleosides comprising 2′-substituted sugar moieties are referred to as 2′-substituted nucleosides. In certain embodiments, a 2′-substituted nucleoside comprises a 2′-substituent group selected from halo, allyl, amino, azido, O—C₁-C₁₀ alkoxy; O—C₁-C₁₀ substituted alkoxy, SH, CN, OCN, CF₃, OCF₃, O-alkyl, S-alkyl, N(Rₘ)-alkyl; O-alkenyl, S-alkenyl, or N(Rₘ)-alkenyl; O-alkynyl, S-alkynyl, N(Rₘ)-alkynyl; O-alkylenyl-O-alkyl, alkynyl, alkaryl, aralkyl, O-alkaryl, O-aralkyl, O(CH₂)₂SCH₃, O-(CH₂)₂-O—N(Rₘ)(Rₙ) or O—CH₂-C(═O)—N(Rₘ)(Rₙ), where each Rₘ and Rₙ is, independently, H, an amino protecting group or substituted or unsubstituted C₁-C₁₀ alkyl. These 2′-substituent groups can be further substituted with one or more substituent groups independently selected from hydroxyl, amino, alkoxy, carboxy, benzyl, phenyl, nitro (NO₂), thiol, thioalkoxy (S-alkyl), halogen, alkyl, aryl, alkenyl and alkynyl.

In certain embodiments, a 2′-substituted nucleoside comprises a 2′-substituent group selected from F, NH₂, N₃, OCF₃, O—CH₃, O(CH₂)₃NH₂, CH2—CH═CH₂, O—CH₂—CH═CH₂, OCH₂CH₂OCH₃, O(CH₂)₂SCH₃, O—(CH₂)₂—O—N(Rₘ)(Rₙ), O(CH₂)₂O(CH₂)₂N(CH₃)₂, and N-substituted acetamide (O—CH₂—C(═O)—N(Rₘ)(Rₙ) where each Rₘ and Rₙ is, independently, H, an amino protecting group or substituted or unsubstituted C₁-C₁₀ alkyl.

In certain embodiments, a 2′-substituted nucleoside comprises a sugar moiety comprising a 2′-substituent group selected from F, OCF₃, O—CH₃, OCH₂CH₂OCH₃, O(CH₂)₂SCH₃, O—(CH₂)₂—O—N(CH₃)₂, —O(CH₂)₂O(CH₂)₂N(CH₃)₂, and O—CH₂—C(═O)—N(H)CH₃.

In certain embodiments, a 2′-substituted nucleoside comprises a sugar moiety comprising a 2′-substituent group selected from F, O—CH₃, and OCH₂CH₂OCH₃.

Certain modifed sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ sugar substituents, include, but are not limited to: —[C(Rₐ)(R_{b})]ₙ-, —[C(Rₐ)(R_{b})]ₙ—O—, —C(RₐR_{b})—N(R)—O— or, —C(RₐR_{b})—O—N(R)—; 4′—CH₂-2′, 4′—(CH₂)₂-2′, 4′—(CH₂)₃-2′, 4′—(CH₂)—O-2′(LNA); 4′—(CH₂)—S-2; 4′-(CH₂)₂—O- 2′(ENA); 4′—CH(CH₃)-—O-2′(cEt) and 4′13 CH(CH₂OCH₃)—O-2′, and analogs thereof (see, e.g., U.S. Pat. No.7,399,845, issued on Jul. 15, 2008); 4′-C(CH₃)(CH₃)—O-2′ and analogs thereof, (see, e.g., WO2009/006478, published Jan. 8, 2009); 4′—CH₂—N(OCH₃)-2′ and analogs thereof (see, e.g., WO2008/150729, published Dec. 11, 2008); 4′—CH₂—O—N(CH₃)-2′ (see, e.g., US2004/0171570, published Sep. 2, 2004); 4′—CH₂—O—N(R)-2′, and 4′—CH₂—N(R)—O—2′-, wherein each R is, independently, H, a protecting group, or C₁-C₁₂ alkyl; 4′—CH₂—N(R)—O—-2′, wherein R is H, C₁-C₁₂ alkyl, or a protecting group (see, U.S. Pat. No. 7,427,672, issued on Sep. 23, 2008); 4′—CH₂—C(H)(CH₃)-2′ (see, e.g., Chattopadhyaya, et al., J. Org. Chem., 2009, 74, 118-134); and 4′—CH₂-C(═CH₂)-2′ and analogs thereof (see, published PCT International Application WO 2008/154401, published on Dec. 8, 2008).

In certain embodiments, such 4′ to 2′ bridges independently comprise from 1 to 4 linked groups independently selected from —[C(Rₐ)(R_{b})]ₙ-, —C(Rₐ)═C(R_{b})—, —C(Rₐ)═N—, —C(═NRₐ)—, —C(═O)—, —C(═S)—, —O—, —Si(Rₐ)₂-, —S(═O)ₓ-, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Nucleosides comprising bicyclic sugar moieties are referred to as bicyclic nucleosides or BNAs. Bicyclic nucleosides include, but are not limited to, (A) α-L-Methyleneoxy (4′—CH₂—O-2′) BNA, (B) β-D-Methyleneoxy (4′-CH₂—O-2′) BNA (also referred to as locked nucleic acid or LNA), (C) Ethyleneoxy (4′—(CH₂)₂—O-2′) BNA, (D) Aminooxy (4′-CH₂—O—N(R)-2′) BNA, (E) Oxyamino (4′—CH₂—N(R)—O-2′) BNA, (F) Methyl(methyleneoxy) (4′—CH(CH₃)—O-2′) BNA (also referred to as constrained ethyl or cEt), (G) methylene-thio (4′—CH₂—S-2′) BNA, (H) methylene-amino (4′—CH2—N(R)-2′) BNA, (I) methyl carbocyclic (4′—CH₂—CH(CH₃)-2′) BNA, and (J) propylene carbocyclic (4′—(CH₂)₃-2′) BNA as depicted below.

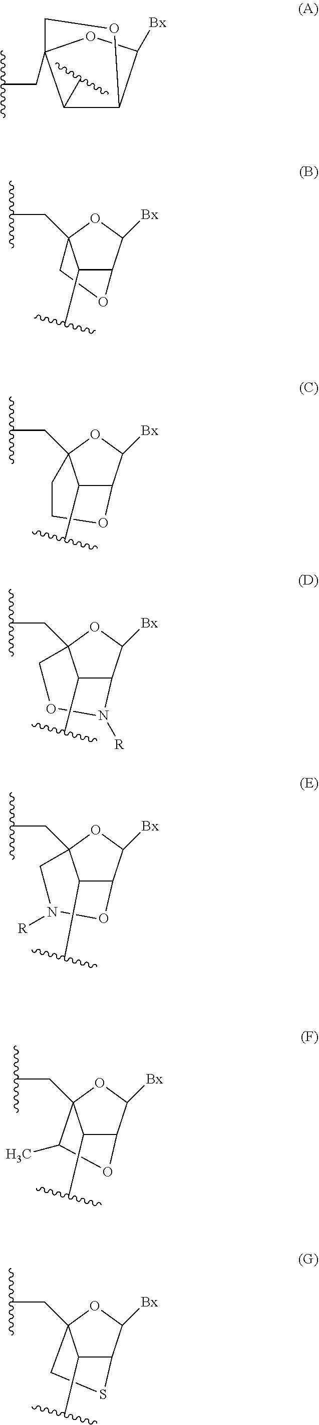

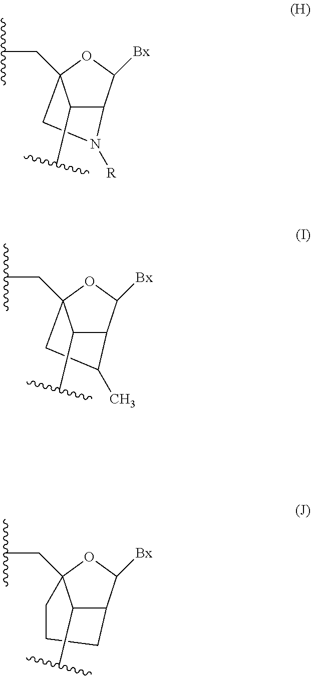

wherein Bx is a nucleobase moiety and R is, independently, H, a protecting group, or C₁-C₁₂ alkyl.

Additional bicyclic sugar moieties are known in the art, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 129(26) 8362-8379 (Jul. 4, 2007); Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; U.S. Pat. Nos. 7,053,207, 6,268,490, 6,770,748, 6,794,499, 7,034,133, 6,525,191, 6,670,461, and 7,399,845; WO 2004/106356, WO 1994/14226, WO 2005/021570, and WO 2007/134181; U.S. Patent Publication Nos. US2004/0171570, US2007/0287831, and US2008/0039618; U.S. patent Ser. Nos. 12/129,154, 60/989,574, 61/026,995, 61/026,998, 61/056,564, 61/086,231, 61/097,787, and 61/099,844; and PCT International Applications Nos. PCT/US2008/064591, PCT/US2008/066154, and PCT/US2008/068922.

In certain embodiments, bicyclic sugar moieties and nucleosides incorporating such bicyclic sugar moieties are further defined by isomeric configuration. For example, a nucleoside comprising a 4′-2′ methylene-oxy bridge, may be in the α-L configuration or in the β-D configuration. Previously, α-L-methyleneoxy (4′—CH₂—O-2′) bicyclic nucleosides have been incorporated into antisense oligonucleotides that showed antisense activity (Frieden et al., Nucleic Acids Research, 2003, 21, 6365-6372).

In certain embodiments, substituted sugar moieties comprise one or more non-bridging sugar substituent and one or more bridging sugar substituent (e.g., 5′-substituted and 4′-2′ bridged sugars). (see, PCT International Application WO 2007/134181, published on Nov. 22, 2007, wherein LNA is substituted with, for example, a 5′-methyl or a 5′-vinyl group).

In certain embodiments, modified sugar moieties are sugar surrogates. In certain such embodiments, the oxygen atom of the naturally occurring sugar is substituted, e.g., with a sulfer, carbon or nitrogen atom. In certain such embodiments, such modified sugar moiety also comprises bridging and/or non-bridging substituents as described above. For example, certain sugar surogates comprise a 4′-sulfer atom and a substitution at the 2′-position (see,e.g., published U.S. Patent Application US2005/0130923, published on Jun. 16, 2005) and/or the 5′ position. By way of additional example, carbocyclic bicyclic nucleosides having a 4′-2′ bridge have been described (see, e.g., Freier et al., Nucleic Acids Research, 1997, 25(22), 4429-4443 and Albaek et al., J. Org. Chem., 2006, 71, 7731-7740).

In certain embodiments, sugar surrogates comprise rings having other than 5-atoms. For example, in certain embodiments, a sugar surrogate comprises a six-membered tetrahydropyran. Such tetrahydropyrans may be further modified or substituted. Nucleosides comprising such modified tetrahydropyrans include, but are not limited to, hexitol nucleic acid (HNA), anitol nucleic acid (ANA), manitol nucleic acid (MNA) (see Leumann, C J. Bioorg. & Med. Chem. (2002) 10:841-854), fluoro HNA (F-HNA), and those compounds having Formula VII:

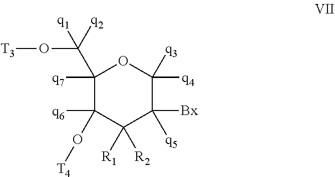

wherein independently for each of said at least one tetrahydropyran nucleoside analog of Formula VII:

Bx is a nucleobase moiety;

T₃ and T₄ are each, independently, an internucleoside linking group linking the tetrahydropyran nucleoside analog to the antisense compound or one of T₃ and T₄ is an internucleoside linking group linking the tetrahydropyran nucleoside analog to the antisense compound and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and

one of R₁ and R₂ is hydrogen and the other is selected from halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, the modified THP nucleosides of Formula VII are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, THP nucleosides of Formula VII are provided wherein one of R₁ and R₂ is F. In certain embodiments, R₁ is fluoro and R₂ is H, R₁ is methoxy and R₂ is H, and R₁ is methoxyethoxy and R₂ is H.

Many other bicyclic and tricyclic sugar and sugar surrogate ring systems are known in the art that can be used to modify nucleosides (see, e.g., review article: Leumann, J. C, Bioorganic & Medicinal Chemistry, 2002, 10, 841-854).

In certain embodiments, sugar surrogates comprise rings having more than 5 atoms and more than one heteroatom. For example nucleosides comprising morpholino sugar moieties and their use in oligomeric compounds has been reported (see for example: Braasch et al., Biochemistry, 2002, 41, 4503-4510; and U.S. Pat. Nos. 5,698,685; 5,166,315; 5,185,444; and 5,034,506). As used here, the term “morpholino” means a sugar surrogate having the following structure:

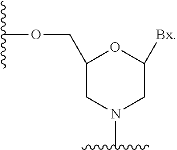

In certain embodiments, morpholinos may be modified, for example by adding or altering various substituent groups from the above morpholino structure. Such sugar surrogates are referred to herein as “modifed morpholinos.”

Combinations of modifications are also provided without limitation, such as 2′-F-5′-methyl substituted nucleosides (see PCT International Application WO 2008/101157 Published on Aug. 21, 2008 for other disclosed 5′, 2′-bis substituted nucleosides) and replacement of the ribosyl ring oxygen atom with S and further substitution at the 2′-position (see published U.S. Patent Application US2005-0130923, published on Jun. 16, 2005) or alternatively 5′-substitution of a bicyclic nucleic acid (see PCT International Application WO 2007/134181, published on Nov. 22, 2007 wherein a 4′-CH₂—O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described (see, e.g., Srivastava et al., J. Am. Chem. Soc. 2007, 129(26), 8362-8379).

### Certain Nucleobases

In certain embodiments, nucleosides of the present invention comprise one or more unmodified nucleobases. In certain embodiments, nucleosides of the present invention comprise one or more modifed nucleobases.

In certain embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and O-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859; those disclosed by Englisch et al., Angewandte Chemie, International Edition, 1991, 30, 613; and those disclosed by Sanghvi, Y. S., Chapter 15, Antisense Research and Applications, Crooke, S. T. and Lebleu, B., Eds., CRC Press, 1993, 273-288.

### Certain Internucleoside Linkages

In certain embodiments, the present invention provides oligomeric compounds comprising linked nucleosides. In such embodiments, nucleosides may be linked together using any internucleoside linkage. The two main classes of internucleoside linking groups are defined by the presence or absence of a phosphorus atom. Representative phosphorus containing internucleoside linkages include, but are not limited to, phosphodiesters (P═O), phosphotriesters, methylphosphonates, phosphoramidate, and phosphorothioates (P═S). Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂-N(CH₃)—N(CH₃)—). Modified linkages, compared to natural phosphodiester linkages, can be used to alter, typically increase, nuclease resistance of the oligomeric compound. In certain embodiments, internucleoside linkages having a chiral atom can be prepared as a racemic mixture, or as separate enantiomers. Representative chiral linkages include, but are not limited to, alkylphosphonates and phosphorothioates. Methods of preparation of phosphorous-containing and non-phosphorous-containing internucleoside linkages are well known to those skilled in the art.

### Certain Motifs

In certain embodiments, the present invention provides oligomeric compounds comprising oligonucleotides. In certain embodiments, such oligonucleotides comprise one or more chemical modification. In certain embodiments, chemically modified oligonucleotides comprise one or more modified nucleosides. In certain embodiments, chemically modified oligonucleotides comprise one or more modified nucleosides comprising modified sugars. In certain embodiments, chemically modified oligonucleotides comprise one or more modified nucleosides comprising one or more modified nucleobases. In certain embodiments, chemically modified oligonucleotides comprise one or more modified internucleoside linkages. In certain embodiments, the chemically modifications (sugar modifications, nucleobase modifications, and/or linkage modifications) define a pattern or motif. In certain embodiments, the patterns of chemical modifications of sugar moieties, internucleoside linkages, and nucleobases are each independent of one another. Thus, an oligonucleotide may be described by its sugar modification motif, internucleoside linkage motif and/or nucleobase modification motif (as used herein, nucleobase modification motif describes the chemical modifications to the nucleobases independent of the sequence of nucleobases).

### Certain Sugar Motifs

In certain embodiments, oligonucleotides comprise one or more type of modified sugar moieties and/or naturally occurring sugar moieties arranged along an oligonucleotide or region thereof in a defined pattern or sugar modification motif. Such motifs may include any of the sugar modifications discussed herein and/or other known sugar modifications.

In certain embodiments, the oligonucleotides comprise or consist of a region having a gapmer sugar modification motif, which comprises two external regions or “wings” and an internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap. In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar modification motifs of the two wings are the same as one another (symmetric gapmer). In certain embodiments, the sugar modification motifs of the 5′-wing differs from the sugar modification motif of the 3′-wing (asymmetric gapmer). In certain embodiments, oligonucleotides comprise 2′-MOE modified nucleosides in the wings and 2′-F modified nucleosides in the gap.

In certain embodiments, oligonucleotides are fully modified. In certain such embodiments, oligonucleotides are uniformly modified. In certain embodiments, oligonucleotides are uniform 2′-MOE. In certain embodiments, oligonucleotides are uniform 2′-F. In certain embodiments, oligonucleotides are uniform morpholino. In certain embodiments, oligonucleotides are uniform BNA. In certain embodiments, oligonucleotides are uniform LNA. In certain embodiments, oligonucleotides are uniform cEt.

In certain embodiments, oligonucleotides comprise a uniformly modified region and additional nucleosides that are unmodified or differently modified. In certain embodiments, the uniformly modified region is at least 5, 10, 15, or 20 nucleosides in length. In certain embodiments, the uniform region is a 2′-MOE region. In certain embodiments, the uniform region is a 2′-F region. In certain embodiments, the uniform region is a morpholino region. In certain embodiments, the uniform region is a BNA region. In certain embodiments, the uniform region is a LNA region. In certain embodiments, the uniform region is a cEt region.

In certain embodiments, the oligonucleotide does not comprise more than 4 contiguous unmodified 2′-deoxynucleosides. In certain circumstances, antisesense oligonucleotides comprising more than 4 contiguous 2′-deoxynucleosides activate RNase H, resulting in cleavage of the target RNA. In certain embodiments, such cleavage is avoided by not having more than 4 contiguous 2′-deoxynucleosides, for example, where alteration of splicing and not cleavage of a target RNA is desired.

### Certain Internucleoside Linkage Motifs

In certain embodiments, oligonucleotides comprise modified internucleoside linkages arranged along the oligonucleotide or region thereof in a defined pattern or modified internucleoside linkage motif. In certain embodiments, internucleoside linkages are arranged in a gapped motif, as described above for sugar modification motif. In such embodiments, the internucleoside linkages in each of two wing regions are different from the internucleoside linkages in the gap region. In certain embodiments the internucleoside linkages in the wings are phosphodiester and the internucleoside linkages in the gap are phosphorothioate. The sugar modification motif is independently selected, so such oligonucleotides having a gapped internucleoside linkage motif may or may not have a gapped sugar modification motif and if it does have a gapped sugar motif, the wing and gap lengths may or may not be the same.

In certain embodiments, oligonucleotides comprise a region having an alternating internucleoside linkage motif In certain embodiments, oligonucleotides of the present invention comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

### Certain Nucleobase Modification Motifs

In certain embodiments, oligonucleotides comprise chemical modifications to nucleobases arranged along the oligonucleotide or region thereof in a defined pattern or nucleobases modification motif. In certain such embodiments, nucleobase modifications are arranged in a gapped motif. In certain embodiments, nucleobase modifications are arranged in an alternating motif In certain embodiments, each nucleobase is modified. In certain embodiments, none of the nucleobases is chemically modified.

In certain embodiments, oligonucleotides comprise a block of modified nucleobases. In certain such embodiments, the block is at the 3′-end of the oligonucleotide. In certain embodiments the block is within 3 nucleotides of the 3′-end of the oligonucleotide. In certain such embodiments, the block is at the 5′-end of the oligonucleotide. In certain embodiments the block is within 3 nucleotides of the 5′-end of the oligonucleotide.

In certain embodiments, nucleobase modifications are a function of the natural base at a particular position of an oligonucleotide. For example, in certain embodiments each purine or each pyrimidine in an oligonucleotide is modified. In certain embodiments, each adenine is modified. In certain embodiments, each guanine is modified. In certain embodiments, each thymine is modified. In certain embodiments, each cytosine is modified. In certain embodiments, each uracil is modified.

In certain embodiments, some, all, or none of the cytosine moieties in an oligonucleotide are 5-methyl cytosine moieties. Herein, 5-methyl cytosine is not a “modified nucleobase.” Accordingly, unless otherwise indicated, unmodified nucleobases include both cytosine residues having a 5-methyl and those lacking a 5 methyl. In certain embodiments, the methylation state of all or some cytosine nucleobases is specified.

### Certain Overall Lengths

In certain embodiments, the present invention provides oligomeric compounds including oligonucleotides of any of a variety of ranges of lengths. In certain embodiments, the invention provides oligomeric compounds or oligonucleotides consisting of X to Y linked nucleosides, where X represents the fewest number of nucleosides in the range and Y represents the largest number of nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, the invention provides oligomeric compounds which comprise oligonucleotides consisting of 8 to 9, 8 to 10, 8 to 11, 8 to 12, 8 to 13, 8 to 14, 8 to 15, 8 to 16, 8 to 17, 8 to 18, 8 to 19, 8 to 20, 8 to 21, 8 to 22, 8 to 23, 8 to 24, 8 to 25, 8 to 26, 8 to 27, 8 to 28, 8 to 29, 8 to 30, 9 to 10, 9 to 11, 9 to 12, 9 to 13, 9 to 14, 9 to 15, 9 to 16, 9 to 17, 9 to 18, 9 to 19, 9 to 20, 9 to 21, 9 to 22, 9 to 23, 9 to 24, 9 to 25, 9 to 26, 9 to 27, 9 to 28, 9 to 29, 9 to 30, 10 to 11, 10 to 12, 10 to 13, 10 to 14, 10 to 15, 10 to 16, 10 to 17, 10 to 18, 10 to 19, 10 to 20, 10 to 21, 10 to 22, 10 to 23, 10 to 24, 10 to 25, 10 to 26, 10 to 27, 10 to 28, 10 to 29, 10 to 30, 11 to 12, 11 to 13, 11 to 14, 11 to 15, 11 to 16,11to 17,11to 18,11to 19,11to 20, 11 to 21,11to 22,11to 23,11to 24,11to 25, 11 to 26,11to 27, 11 to 28, 11 to 29, 11 to 30, 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides. In embodiments where the number of nucleosides of an oligomeric compound or oligonucleotide is limited, whether to a range or to a specific number, the oligomeric compound or oligonucleotide may, nonetheless further comprise additional other substituents. For example, an oligonucleotide comprising 8-30 nucleosides excludes oligonucleotides having 31 nucleosides, but, unless otherwise indicated, such an oligonucleotide may further comprise, for example one or more conjugates, terminal groups, or other substituents. In certain embodiments, a gapmer oligonucleotide has any of the above lengths.

### Certain Oligonucleotides

In certain embodiments, oligonucleotides of the present invention are characterized by their sugar motif, internucleoside linkage motif, nucleobase modification motif and overall length. In certain embodiments, such parameters are each independent of one another. Thus, each internucleoside linkage of an oligonucleotide having a gapmer sugar motif may be modified or unmodified and may or may not follow the gapmer modification pattern of the sugar modifications. Thus, the internucleoside linkages within the wing regions of a sugar-gapmer may be the same or different from one another and may be the same or different from the internucleoside linkages of the gap region. Likewise, such sugar-gapmer oligonucleotides may comprise one or more modified nucleobase independent of the gapmer pattern of the sugar modifications. Herein if a description of an oligonucleotide or oligomeric compound is silent with respect to one or more parameter, such parameter is not limited. Thus, an oligomeric compound described only as having a gapmer sugar motif without further description may have any length, internucleoside linkage motif, and nucleobase modification motif. Unless otherwise indicated, all chemical modifications are independent of nucleobase sequence.

### Certain Conjugate Groups

In certain embodiments, oligomeric compounds comprise oligonucleotides modified by attachment of one or more conjugate groups. In general, conjugate groups modify one or more properties of the attached oligomeric compound including but not limited to pharmacodynamics, pharmacokinetics, stability, binding, absorption, cellular distribution, cellular uptake, charge and clearance. Conjugate groups are routinely used in the chemical arts and are linked directly or via an optional conjugate linking moiety or conjugate linking group to a parent compound such as an oligomeric compound, such as an oligonucleotide. Conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

In certain embodiments, conjugate groups are directly attached to oligonucleotides in oligomeric compounds. In certain embodiments, conjugate groups are attached to oligonucleotides by a conjugate linking group. In certain such embodiments, conjugate linking groups, including, but not limited to, bifunctional linking moieties such as those known in the art are amenable to the compounds provided herein. Conjugate linking groups are useful for attachment of conjugate groups, such as chemical stabilizing groups, functional groups, reporter groups and other groups to selective sites in a parent compound such as for example an oligomeric compound. In general a bifunctional linking moiety comprises a hydrocarbyl moiety having two functional groups. One of the functional groups is selected to bind to a parent molecule or compound of interest and the other is selected to bind essentially any selected group such as chemical functional group or a conjugate group. In some embodiments, the conjugate linker comprises a chain structure or an oligomer of repeating units such as ethylene glycol or amino acid units. Examples of functional groups that are routinely used in a bifunctional linking moiety include, but are not limited to, electrophiles for reacting with nucleophilic groups and nucleophiles for reacting with electrophilic groups. In some embodiments, bifunctional linking moieties include amino, hydroxyl, carboxylic acid, thiol, unsaturations (e.g., double or triple bonds), and the like.

In certain embodiments, conjugate groups are at the 3′-end of an oligonucleotide of an oligomeric compound. In certain embodiments, conjugate groups are near the 3′-end. In certain embodiments, conjugates are attached at the 3′end of an oligomeric compound, but before one or more terminal group nucleosides. In certain embodiments, conjugate groups are placed within a terminal group. In certain embodiments, the present invention provides oligomeric compounds. In certain embodiments, oligomeric compounds comprise an oligonucleotide. In certain embodiments, an oligomeric compound comprises an oligonucleotide and one or more conjugate and/or terminal groups. Such conjugate and/or terminal groups may be added to oligonucleotides having any of the chemical motifs discussed above. Thus, for example, an oligomeric compound comprising an oligonucleotide having region of alternating nucleosides may comprise a terminal group.

### Antisense Compounds

In certain embodiments, oligomeric compounds of the present invention are antisense compounds. Such antisense compounds are capable of hybridizing to a target nucleic acid, resulting in at least one antisense activity. In certain embodiments, antisense compounds specifically hybridize to one or more target nucleic acid. In certain embodiments, a specifically hybridizing antisense compound has a nucleobase sequence comprising a region having sufficient complementarity to a target nucleic acid to allow hybridization and result in antisense activity and insufficient complementarity to any non-target so as to avoid non-specific hybridization to any non-target nucleic acid sequences under conditions in which specific hybridization is desired (e.g., under physiological conditions for in vivo or therapeutic uses, and under conditions in which assays are performed in the case of in vitro assays).

In certain embodiments, the present invention provides antisense compounds comprising oligonucleotides that are fully complementary to the target nucleic acid over the entire length of the oligonucleotide. In certain embodiments, oligonucleotides are 99% complementary to the target nucleic acid. In certain embodiments, oligonucleotides are 95% complementary to the target nucleic acid. In certain embodiments, such oligonucleotides are 90% complementary to the target nucleic acid.

In certain embodiments, such oligonucleotides are 85% complementary to the target nucleic acid. In certain embodiments, such oligonucleotides are 80% complementary to the target nucleic acid. In certain embodiments, an antisense compound comprises a region that is fully complementary to a target nucleic acid and is at least 80% complementary to the target nucleic acid over the entire length of the oligonucleotide. In certain such embodiments, the region of full complementarity is from 6 to 14 nucleobases in length.

In certain embodiments, oligomeric compounds having any motif described herein have a nucleobase sequence complementary to intron 7 of SMN2. Certain such nucleobase sequences are exemplified in the non-limiting table below.

| Sequence | Length | SEQ ID |
|---|---|---|
| TGCTGGCAGACTTAC | 15 | 3 |
| CATAATGCTGGCAGA | 15 | 4 |
| TCATAATGCTGGCAG | 15 | 5 |
| TTCATAATGCTGGCA | 15 | 6 |
| TTTCATAATGCTGGC | 15 | 2 |
| ATTCACTTTCATAATGCTGG | 20 | 7 |
| TCACTTTCATAATGCTGG | 18 | 1 |
| CTTTCATAATGCTGG | 15 | 8 |
| TCATAATGCTGG | 12 | 9 |
| ACTTTCATAATGCTG | 15 | 10 |
| TTCATAATGCTG | 12 | 11 |
| CACTTTCATAATGCT | 15 | 12 |
| TTTCATAATGCT | 12 | 13 |
| TCACTTTCATAATGC | 15 | 14 |
| CTTTCATAATGC | 12 | 15 |
| TTCACTTTCATAATG | 15 | 16 |
| ACTTTCATAATG | 12 | 17 |
| ATTCACTTTCATAAT | 15 | 18 |
| CACTTTCATAAT | 12 | 19 |
| GATTCACTTTCATAA | 15 | 20 |
| TCACTTTCATAA | 12 | 21 |
| TTCACTTTCATA | 12 | 22 |
| ATTCACTTTCAT | 12 | 23 |
| AGTAAGATTCACTTT | 15 | 24 |

Antisense compounds for use in the present invention can be used to modulate the expression of SMN2 in a subject, such as a human. In certain embodiments, the subject has spinal muscular atrophy. In certain such subjects, the SMN1 gene is absent or otherwise fails to produce sufficient amounts of functional SMN protein. In certain embodiments, the antisense compounds of the present invention effectively modulate splicing of SMN2, resulting in an increase in exon 7 inclusion in SMN2 mRNA and ultimately in SMN2 protein that includes the amino acids corresponding to exon 7. Such alternate SMN2 protein resembles wild-type SMN protein. Antisense compounds for use in the present invention that effectively modulate expression of SMN2 mRNA or protein products of expression are active antisense compounds.

### Pharmaceutical Compositions

In certain embodiments, the present invention provides pharmaceutical compositions comprising one or more antisense compound. In certain embodiments, such pharmaceutical composition comprises a sterile saline solution and one or more antisense compound. In certain embodiments, such pharmaceutical composition consists of a sterile saline solution and one or more antisense compound.

In certain embodiments, antisense compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments antisense compounds, can be utilized in pharmaceutical compositions by combining such oligomeric compounds with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in certain embodiments, employed in the methods described herein is a pharmaceutical composition comprising an antisense compound and a pharmaceutically acceptable diluent. In certain embodiments, the pharmaceutically acceptable diluent is PBS.

Pharmaceutical compositions comprising antisense compounds encompass any pharmaceutically acceptable salts, esters, or salts of such esters. In certain embodiments, pharmaceutical compositions comprising antisense compounds comprise one or more oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

### Certain Subjects

In certain embodiments, a subject has one or more indicator of SMA. In certain embodiments, the subject has reduced electrical activity of one or more muscles. In certain embodiments, the subject has a mutant SMN1 gene. In certain embodiment, the subject's SMN1 gene is absent or incapable of producing functional SMN protein. In certain embodiments, the subject is diagnosed by a genetic test. In certain embodiments, the subject is identified by muscle biopsy. In certain embodiments, a subject is unable to sit upright. In certain embodiments, a subject is unable to stand or walk. In certain embodiments, a subject requires assistance to breathe and/or eat. In certain embodiment, a subject is identified by electrophysiological measurement of muscle and/or muscle biopsy.

In certain embodiments, the subject has SMA type I. In certain embodiments, the subject has SMA type II. In certain embodiments, the subject has SMA type III. In certain embodiments, the subject is diagnosed as having SMA in utero. In certain embodiments, the subject is diagnosed as having SMA within one week after birth. In certain embodiments, the subject is diagnosed as having SMA within one month of birth. In certain embodiments, the subject is diagnosed as having SMA by 3 months of age. In certain embodiments, the subject is diagnosed as having SMA by 6 months of age. In certain embodiments, the subject is diagnosed as having SMA by 1 year of age. In certain embodiments, the subject is diagnosed as having SMA between 1 and 2 years of age. In certain embodiments, the subject is diagnosed as having SMA between 1 and 15 years of age. In certain embodiments, the subject is diagnosed as having SMA when the subject is older than 15 years of age.

### Certain Treatment Methods

In certain embodiments, a subject having SMA is administered a GF/IGF axis modulator. In certain embodiments, a subject having SMA is administered a GF/IGF axis molecule. In certain embodiments, a subject having SMA is administered a GF/IGF axis modulator and an antisense compound that modulates splicing of SMN2. In certain embodiments, a subject having SMA is administered a GF/IGF axis molecule and an antisense compound that modulates splicing of SMN2.

In certain embodiments, the first dose of at least one GF/IGF axis modulator is administered in utero. In certain such embodiments, the first dose is administered before complete development of the blood-brain-barrier. In certain embodiments, the first dose is administered in utero after formation of the blood-brain-barrier. In certain embodiments, the first dose is administered to the subject in utero systemically. In certain embodiments, the first dose is administered to the CSF.

In certain embodiments, the first dose of at least one GF/IGF axis modulator is administered when the subject is less than one week old. In certain embodiments, the first dose of at least one GF/IGF axis modulator is administered when the subject is less than one month old. In certain embodiments, the first dose at least one GF/IGF axis modulator is administered when the subject is less than 3 months old. In certain embodiments, the first dose of at least one GF/IGF axis modulator is administered when the subject is less than 6 months old. In certain embodiments, the first dose of at least one GF/IGF axis modulator is administered when the subject is less than one year old. In certain embodiments, the first dose of at least one GF/IGF axis modulator is administered when the subject is less than 2 years old. In certain embodiments, the first dose at least one GF/IGF axis modulator is administered when the subject is less than 15 years old. In certain embodiments, the first dose of at least one GF/IGF axis modulator is administered when the subject is older than 15 years old.

Certain methods of administration are exemplified in the following non-limiting table.

| GF/IGF axis modulator | | Antisense compound | |
|---|---|---|---|
| Identity | Route | Identity | Route |
| IGFALS | Systemic | Uniform MOE ASO | IT |
| IGFALS | Systemic | Uniform MOE ASO | Systemic |
| | | | and IT |
| IGFALS | Systemic | None | NA |
| IGFALS | Systemic | Uniform MOE ASO | Systemic |
| | and IT | | and IT |
| IGFALS | Systemic | Uniform MOE ASO | Systemic |
| gene | | | and IT |
| IGFALS | Systemic | Uniform MOE ASO | Systemic |
| gene | and IT | | and IT |
| IGFALS | Systemic | Uniform morpholino ASO | IT |
| IGFALS | Systemic | Uniform morpholino ASO | Systemic |
| | | | and IT |
| IGF-1 | Systemic | Full MOE ASO | IT |
| | and IT | | |
| IGF-1 | Systemic | Full MOE ASO | Systemic |
| | | | and IT |

The above table is non-limiting and solely to illustrate how the components of the present invention may be independently manipulated. Moreover, in certain embodiments, the antisense compound may be substituted with other strategies for increasing SMN protein, such as gene therapy encoding SMN1.

Since IGF-1 is an important factor in neurons, the present invention also provides methods of treating neurodegenerative conditions other than SMA. For example, in certain embodiments, the subject has Alzheimer's disease or amyotrophic lateral sclerosis.

### Co-Administration

In certain embodiments, at least one GF/IGF axis modulator (with or without an antisense compound or gene therapy for increasing SMN protein systemically and/or in the CNS) is co-administered with at least one other pharmaceutical composition for treating SMA and/or for treating one or more symptom associated with SMA. In certain embodiments, such other pharmaceutical composition is selected from trichostatin-A, valproic acid, riluzole, hydroxyurea, and a butyrate or butyrate derivative. In certain embodiments, pharmaceutical compositions of the present invention are co-administered with trichostatin A. In certain embodiments, pharmaceutical compositions of the present invention are co-administered with a derivative of quinazoline, for example as described in Thurmond, et al., J. Med Chem. 2008, 51, 449-469. In certain embodiments, a pharmaceutical composition of the present invention and at least one other pharmaceutical composition are co-administered at the same time. In certain embodiments, a pharmaceutical composition of the present invention and at least one other pharmaceutical composition are co-administered at different times.

In certain embodiments, pharmaceutical compositions of the present invention are co-administered with a gene therapy agent. In certain such embodiments, the gene therapy agent is administered to the CSF and the pharmaceutical composition of the present invention is administered systemically. In certain such embodiments, the gene therapy agent is administered to the CSF and the pharmaceutical composition of the present invention is administered to the CSF and systemically. In certain embodiments, a pharmaceutical composition of the present invention and a gene therapy agent are co-administered at the same time. In certain embodiments, a pharmaceutical composition of the present invention and a gene therapy agent are co-administered at different times. Certain gene therapy approaches to SMA treatment have been reported (e.g., Coady et al., PLoS ONE 2008 3(10): e3468; Passini et al., J Clin Invest 2010 Apr. 1, 120(4): 1253-64).

In certain embodiments, pharmaceutical compositions of the present invention are co-administered with at least one other therapy for SMA. In certain embodiments, such other therapy for SMA is surgery. In certain embodiments, such other therapy is physical therapy, including, but not limited to exercises designed to strengthen muscles necessary for breathing, such as cough therapy. In certain embodiments, other therapy is a physical intervention, such as a feeding tube or device for assisted breathing.

In certain embodiments, pharmaceutical compositions of the present invention are co-administered with one or more other pharmaceutical compositions that reduce an undesired side-effect of the pharmaceutical compositions of the present invention.

### Nonlimiting Disclosure and Incorporation by Reference

While certain compounds, compositions and methods described herein have been described with specificity in accordance with certain embodiments, the following examples serve only to illustrate the compounds described herein and are not intended to limit the same. Each of the references, GenBank accession numbers, and the like recited herein is hereby incorporated by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH for the natural 2′-H of DNA) or as an RNA having a modified base (thymine (methylated uracil) for natural uracil of RNA).

Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified bases, such as “AT^{me}CGAUCG,” wherein ^{me}C indicates a cytosine base comprising a methyl group at the 5-position.

### EXAMPLE 1

### Antisense Compounds Targeting SMN2

The following oligonucleotide was synthesized using standard techniques previously reported.

| Reference # | Sequence | Length | Chemistry | SEQ ID |
|---|---|---|---|---|
| ISIS396443 | TCACTTTCATAATGCTGG | 18 | Full 2'-MOE; full PS | 1 |

PS = phosphorothioate intemucleoside linkages

### EXAMPLE 2

### Smn-/-SMN Transgenic Mice

Experiments were performed in a SMA type III mouse model described previously. Riessland, M. et al., SAHA ameliortates the SMA phenotype in two mouse models for spinal muscular atropy. Hum Mol Genet 19, 1492-1506 (2010).

### EXAMPLE 3

### Administration of ISIS 396443

ISIS-396443 was administered to SMA mice as summarized in the table below. Median survival is reported in days or months. Abbreviation: d, days; m, months (>13 months survival indicates that mice were still alive at the time of this filing); SMA, SMA mice; Het, heterozygous mice.

| | | | | SC at P0-P3 | | SC at P5-P7 | | | | | |
|---|---|---|---|---|---|---|---|---|---|---|---|
| | | | ICV at | (2 shots) | | (2 shots) | | Median | Mean | Mice alive | |
| Group Name | Mice | n | P1 | P0-P1 | P2-P3 | P5 | P7 | survival | survival | n | age |

Comparison of ICV to systemic administration

| | | | | | | | | | | | | | |
|---|---|---|---|---|---|---|---|---|---|---|---|---|---|
| SMA-ICV | SMA | 14 | 20 μg | | | | | 16 | d | 17 + 5 | d | 0 | |
| SMA-ICV-Con | SMA | 18 | 0 μg | | | | | 10 | d | 10 + 2 | d | 0 | |
| Het-ICV | Het | 15 | 20 μg | | | | | >14 | m | >14 | m | | |
| SMA-SC | SMA | 12 | | 50 μg/g | 50 μg/g | | | 108 | d | | | 1 | 13 m |
| SMA-ICV-SC | SMA | 18 | 20 μg | 50 μg/g | 50 μg/g | | | 173 | d | | | 2 | 15 m |
| SMA-SC-SC | SMA | 14 | | 50 μg/g | 50 μg/g | 50 μg/g | 50 μg/g | 137 | d | | | 2 | 14 m |
| SMA-SC-Con | SMA | 26 | | 0 μg | 0 μg | | | 9 | d | 10 + 2 | d | | |
| ICV-SC-Con | SMA | 15 | 0 μg | 0 μg | 0 μg | | | 10 | d | 10 + 2 | d | 0 | |
| Het-SC | Het | 12 | | 50 μg/g | 50 μg/g | | | >14 | m | >14 | m | | |
| Het-ICV-SC | Het | 13 | 20 μg | 50 μg/g | 50 μg/g | | | >14 | m | >14 | m | | |

Dose-response study

| | | | | | | | | | | |
|---|---|---|---|---|---|---|---|---|---|---|
| SC40 | SMA | 26 | 40 μg/g | 40 μg/g | 84 | d | | | 1 | 13 m |
| SC80 | SMA | 18 | 80 μg/g | 80 μg/g | 170 | d | | | 2 | 13 m |
| SC160 | SMA | 14 | 160 μg/g | 160 μg/g | 248 | d | | | 3 | 13 m |
| SC-Saline | SMA | 23 | 0 μg/g | 0 μg/g | 10 | d | 10 + 2 | d | 0 | |
| SC160-Het | Het | 18 | 160 μg/g | 160 μg/g | >13 | m | >13 | m | | |
| SC-Sal-Het | Het | 18 | 0 μg/g | 0 μg/g | >13 | m | >13 | m | | |

Late rescue

| | | | | | | | | | |
|---|---|---|---|---|---|---|---|---|---|
| SC-late | SMA | 17 | 100 μg/g | 100 μg/g | 16 | d | 24 + 28 | d | 0 |
| Late-Con | SMA | 15 | 0 μg | 0 μg | 11 | d | 11 + 2 | d | 0 |

IP injection instead of SC injection

| | | | | | | | | | | |
|---|---|---|---|---|---|---|---|---|---|---|
| IP80* | SMA | 16 | 80 μg/g | 80 μg/g | 118 | d | | | 5 | 11 m |
| IP-Saline | SMA | 12 | 0 μg/g | 0 μg/g | 11 | d | 10 + 2 | d | 0 | |
| IP-Het | Het | 14 | 80 μg/g | 80 μg/g | >11 | m | >11 | m | | |

Other treatments

| | | | | | | | | | | |
|---|---|---|---|---|---|---|---|---|---|---|
| ICV-E-0** | SMA | 12 | 0 μg | | | 11 | d | 10 + 2 | d | 0 |
| ICV-E-5** | SMA | 16 | 5 μg | | | 10 | d | 10 + 2 | d | 0 |
| ICV-E-20** | SMA | 26 | 20 μg | | | 12 | d | 14 + 10 | d | 0 |
| ICV-5 | SMA | 16 | 5 μg | | | 10 | d | 10 + 2 | d | 0 |
| ICV-10 | SMA | 15 | 10 μg | | | 11 | d | 12 + 3 | d | 0 |
| ICV- | SMA | 13 | 20 μg | | | 10 | d | 10 + 2 | d | 0 |
| Mismatch^{Δ} | | | | | | | | | | |
| SC-Mismatch^{Δ} | SMA | 17 | | 160 μg/g | 160 μg/g | 10 | d | 10 + 2 | d | 0 |

*IP injection in P0-P3 neonates (IP80) sometimes causes peritoneal fluid including ASO, to leak out, which might explain reduced median survival compared to group SC80.
**ICV injection of ASP-10-27 at embryonic dat 15-16 instead of P1.
^{Δ}ICV or SC injection of a mismatch control ASO11.

### EXAMPLE 3

### Evaluation of Circulating IGF-1

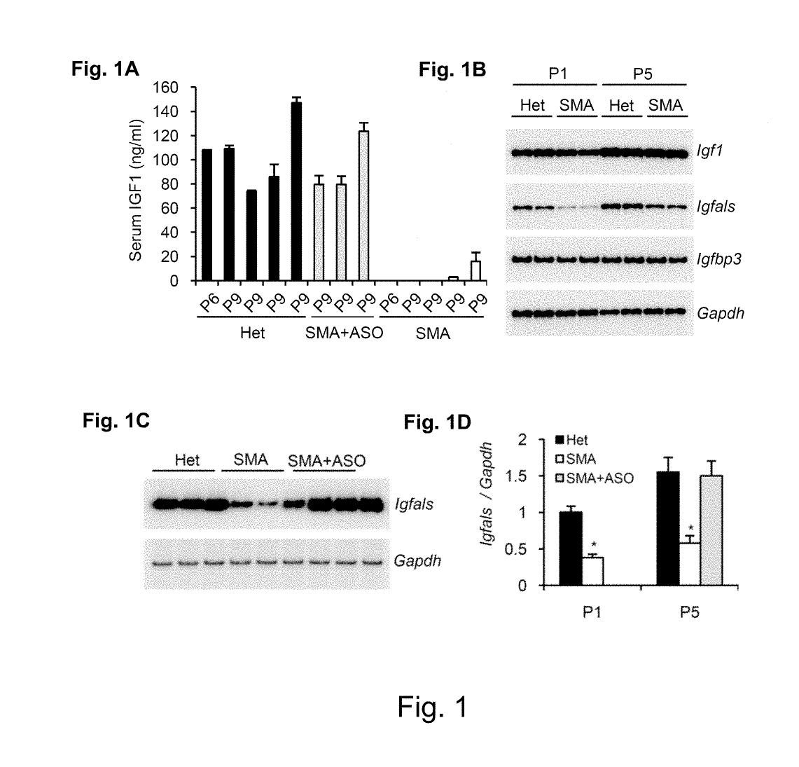

Samples from mice in Example 2 were evaluated for IGF-1 by ELISA assay. Serum samples from day P6-P9 from heterozygous mice (normal phenotype) and SMA mice treated with ISIS-396443 at day 0 had serum levels>60 ng/ml. Serum IGF-1 levels from untreated SMA mice were less than 20 ng/ml. Results are summarized in the graph at FIG. 1a.

### EXAMPLE 4

### Evaluation of GH/IGF-1 Axis RNA

Liver tissue from the SMA mice and heterozygous mice was collected and RNA was extracted using standard techniques. RT-PCR showed that mRNA encoding IGF-1 and IGFBP3 were not reduced in the SMA mice, but mRNA encoding IGFALS was reduced in the SMA mice. This experiment was repeated, adding an ASO treated group. ASO treatment results in normal IGFALS levels. These results are shown in FIGS. 1b-d.

1-70. (canceled)

71. A method of modulating the GF/IGF-1 axis, comprising administering at least one GF/IGF-1 axis modulator, wherein the GF/IGF-1 axis modulator is a GF/IGF-1 axis molecule selected from among: IGF-1 and insulin-like growth factor binding acid labile subunit (IGFALS), to a human subject having spinal muscular atrophy (SMA).

72. The method of claim 71, wherein at least one GF/IGF-1 axis modulator is IGF-binding-protein acid labile subunit (IGFALS).

73. The method of claim 71, wherein at least one GF/IGF-1 axis modulator is IGF-1.

74. The method of claim 71, wherein the at least one GF/IGF-1 axis modulator is administered systemically.

75. The method of claim 71, wherein at least one growth hormone axis modulator is administered by intraperitoneal injection.

76. The method of claim 71, wherein at least one growth hormone axis modulator is administered by subcutaneous injection.

77. The method of claim 71, wherein at least one growth hormone axis modulator is administered by intramuscular injection.

78. The method of claim 71, wherein at least one growth hormone axis modulator is administered into the cerebrospinal fluid.

79. The method of claim 71, comprising administering at least one antisense oligonucleotide to the subject having spinal muscular atrophy.

80. The method of claim 79, wherein the antisense compound comprises an antisense oligonucleotide complementary to a nucleic acid encoding human SMN2.

81. The method of claim 80, wherein the oligonucleotide is complementary to a portion of intron 7 of the nucleic acid encoding human SMN2.

82. The method of claim 80, wherein the antisense oligonucleotide is at least 90% complementary to the nucleic acid encoding human SMN2.

83. The method of claim 80, wherein the antisense oligonucleotide is fully complementary to the nucleic acid encoding human SMN2.

84. The method of claim 80, wherein the oligonucleotide has a nucleobase sequence comprising at least 10 contiguous nucleobases of the nucleobase sequence SEQ ID NO: 1.

85. The method of claim 80, wherein the oligonucleotide has a nucleobase sequence comprising at least 15 contiguous nucleobases of the nucleobase sequence SEQ ID NO: 1.

86. The method of claim 80, wherein the oligonucleotide has a nucleobase sequence comprising the nucleobase sequence SEQ ID NO: 1.

87. The method of claim 80, wherein the oligonucleotide has a nucleobase sequence consisting of the nucleobase sequence SEQ ID NO: 1.

88. The method of claim 80, wherein at least one nucleoside of the antisense oligonucleotide comprises a modified sugar moiety.

89. The method of claim 88, wherein the at least one modified sugar moiety comprises a 2′-methoxyethyl sugar moiety.

90. The method of claim 88, wherein essentially each nucleoside of the antisense oligonucleotide comprises a modified sugar


SEQUENCE LISTING (24 sequences)

SEQ ID NO: 1 - DNA, 18 bp, Artificial sequence
  Synthetic oligonucleotide
tcactttcataatgctgg

SEQ ID NO: 2 - DNA, 15 bp, Artificial sequence
  Synthetic oligonucleotide
tttcataatgctggc

SEQ ID NO: 3 - DNA, 15 bp, Artificial sequence
  Synthetic oligonucleotide
tgctggcagacttac

SEQ ID NO: 4 - DNA, 15 bp, Artificial sequence
  Synthetic oligonucleotide
cataatgctggcaga

SEQ ID NO: 5 - DNA, 15 bp, Artificial sequence
  Synthetic oligonucleotide
tcataatgctggcag

SEQ ID NO: 6 - DNA, 15 bp, Artificial sequence
  Synthetic oligonucleotide
ttcataatgctggca

SEQ ID NO: 7 - DNA, 20 bp, Artificial sequence
  Synthetic oligonucleotide
attcactttcataatgctgg

SEQ ID NO: 8 - DNA, 15 bp, Artificial sequence
  Synthetic oligonucleotide
ctttcataatgctgg

SEQ ID NO: 9 - DNA, 12 bp, Artificial sequence
  Synthetic oligonucleotide
tcataatgctgg

SEQ ID NO: 10 - DNA, 15 bp, Artificial sequence
  Synthetic oligonucleotide
actttcataatgctg

SEQ ID NO: 11 - DNA, 12 bp, Artificial sequence
  Synthetic oligonucleotide
ttcataatgctg

SEQ ID NO: 12 - DNA, 15 bp, Artificial sequence
  Synthetic oligonucleotide
cactttcataatgct

SEQ ID NO: 13 - DNA, 12 bp, Artificial sequence
  Synthetic oligonucleotide
tttcataatgct

SEQ ID NO: 14 - DNA, 15 bp, Artificial sequence
  Synthetic oligonucleotide
tcactttcataatgc

SEQ ID NO: 15 - DNA, 12 bp, Artificial sequence
  Synthetic oligonucleotide
ctttcataatgc

SEQ ID NO: 16 - DNA, 15 bp, Artificial sequence
  Synthetic oligonucleotide
ttcactttcataatg

SEQ ID NO: 17 - DNA, 12 bp, Artificial sequence
  Synthetic oligonucleotide
actttcataatg

SEQ ID NO: 18 - DNA, 15 bp, Artificial sequence
  Synthetic oligonucleotide
attcactttcataat

SEQ ID NO: 19 - DNA, 12 bp, Artificial sequence
  Synthetic oligonucleotide
cactttcataat

SEQ ID NO: 20 - DNA, 15 bp, Artificial sequence
  Synthetic oligonucleotide
gattcactttcataa

SEQ ID NO: 21 - DNA, 12 bp, Artificial sequence
  Synthetic oligonucleotide
tcactttcataa

SEQ ID NO: 22 - DNA, 12 bp, Artificial sequence
  Synthetic oligonucleotide
ttcactttcata

SEQ ID NO: 23 - DNA, 12 bp, Artificial sequence
  Synthetic oligonucleotide
attcactttcat

SEQ ID NO: 24 - DNA, 15 bp, Artificial sequence
  Synthetic oligonucleotide
agtaagattcacttt